(19) United States
(12) Patent Application Publication
(10) Pub. No.: US 2015/0265458 A1
(43) Pub. Date: Sep. 24, 2015

(54) SYSTEM AND METHOD FOR THERAPEUTIC MANAGEMENT OF UNPRODUCTIVE COUGH

(71) Applicant: Circuit Therapeutics, Inc., Menlo Park, CA (US)

(72) Inventors: Dan Andersen, Menlo Park, CA (US); Griffith Roger Thomas, Burlingame, CA (US)

(73) Assignee: Circuit Therapeutics, Inc., Menlo Park, CA (US)

(21) Appl. No.: 14/629,416

(22) Filed: Feb. 23, 2015

Related U.S. Application Data
(60) Provisional application No. 61/943,210, filed on Feb. 21, 2014.

(51) Int. Cl. A61F 7/00

(52) U.S. Cl. CPC A61F 7/007; A61F 2007/0071; A61F 2007/0077; A61F 2007/0018; A61F 2007/0095; A61F 2007/0094

(57) ABSTRACT

One embodiment is directed to a system for managing unproductive cough in a patient, comprising: an applicator comprising a resistive heating element and being configured to be positioned adjacent a portion of a targeted nerve tissue for treatment; a power source configured to provide electrical current to the resistive heating element; and a current controller operatively coupled to the power source and configured to raise the temperature of the portion of the targeted nerve tissue to inhibit nerve conduction.

### RELATED APPLICATION DATA

The present application claims priority to U.S. Provisional Application Ser. No. 61/943,210, filed Feb. 21, 2014. The foregoing application is hereby incorporated by reference into the present application in its entirety.

### FIELD OF THE INVENTION

The present invention relates generally to chronic cough and, in particular, to an implantable configuration for providing warming of cervical vagus nerves for the treatment of chronic cough.

### BACKGROUND

The cough reflex is one of several defensive reflexes that serve to protect the airways from the potentially damaging effects of inhaled particulate matter, aeroallergens, pathogens, aspirate and accumulated secretions. In some airways diseases, cough may become excessive and non-productive, and is potentially harmful to the airway mucosa.

As described in the review entitled Epidemiology of Cough by Alyn Morrice in 2002, (Chung, K., W. J G, et al., Eds. (2003). Cough: Causes, Mechanisms and Therapy. Malden, Mass., Blackwell Publishing Lld.; incorporated by reference herein in its entirety) cough is a universal experience common to us all. It is also the commonest symptom for which medical advice is sought. For the purpose of classification cough may be divided into defined, acute, self-limiting episodes and chronic persistent cough. This distinction is clinically useful since the aetiology of the two syndromes is very different. An arbitrary cut-off of 8 weeks is taken to separate acute from chronic cough.

The Three Common Causes of Chronic Cough.

All of the reported series from tertiary referral centres identify the same three common causes of cough. This diagnostic triad underlies the vast majority of chronic cough seen within the population. The problem of the high morbidity from chronic cough is the failure of doctors, both generalists and specialists, to recognize that cough as an isolated symptom may be generated from any of three anatomical areas.

Cough-Predominant Asthma

The term cough-predominant asthma has been introduced to illustrate that cough may be one facet of an asthma syndrome which is variously represented in individual patients. In classic asthma where bronchoconstriction, and conversely bronchodilator response, can be demonstrated cough may be an additional and important feature. However, cough as an isolated symptom without bronchoconstriction or breathlessness, but with the characteristic pathological features of asthmatic airway inflammation, is the other end of the spectrum. This so-called cough variant asthma is merely one end of a continuum. The term cough-predominant asthma may be preferred since this terminology includes patients in whom the major problem is cough but who also illustrate some or all of the other features of classic asthma.

Between a quarter and a third of patients presenting to a tertiary referral center with chronic cough will be suffering from cough-predominant asthma. This rate of detection probably does not reflect the prevalence of cough-predominant asthma since many patients, particularly those who have features of classic asthma, are diagnosed and treated in the community. Indeed it is unusual for patients with chronic cough to be seen in tertiary clinics who have not had an unsuccessful trial of inhaled medication. The reasons for failure of therapy, even when the underlying diagnosis is of cough-predominant asthma, are all those usually associated with poor asthma control: compliance, poor inhaler technique, inappropriate choice of device, etc. In addition there are other features of cough-predominant asthma, which unless recognized, lead to failure of therapy. Clearly the usual diagnostic measures of reversibility testing or home peak flow monitoring are frequently unhelpful. Even methacholine challenge may not identify patients who respond adequately to corticosteroid therapy since those with eosinophilic bronchitis are not hypersensitive. Whilst sputum examination in expert hands clearly has a role the methodological difficulties obviate its routine use. Ultimately, the diagnosis and therefore prevalence of cough-predominant asthma rests on the use of a therapeutic trial of antiasthma medication. Here again the differences between cough-predominant asthma and classic asthma may lead to confusion. Since bronchospasm may only be a minor feature or even absent, add-on therapy with long-acting β-agonists rarely proves successful and leukotriene antagonists may be the preferred add-on therapy. The response to leukotriene antagonists may illustrate the hypothesized role of lipoxygenase products in the direct modulation of the putative VR1 cough receptor. Ultimately, diagnosis of cough-predominant asthma may rely on the demonstration of a response to parenteral steroids.

The Oesophagus and Cough

A considerable portion of patients presenting with chronic cough have a disorder of the oesophagus. It is poorly recognized by many physicians, yet cough as the sole presentation of gastro-oesophageal reflux has been well described. In addition to reflux it is becoming increasingly clear that a number of oesophageal disorders, broadly classified as dysmotility and including abnormal peristalsis and abnormal lower oesophageal sphincter tone, may give rise to cough. That acid reflux alone is not the cause of cough in oesophageal disease explains the partial response seen in many patients with even high doses of proton pump inhibitors. As with other causes of cough, diagnosis may be difficult because there can be few clues from the history. However, whilst there is some disagreement, in individual patients there may be a strong association with other symptoms, particularly heartburn. More unusual characteristics such as an association with hoarseness, choking sensation and postnasal symptoms are increasingly recognized as being part of a reflux phenomenon by ENT specialists. Indeed, a striking reduction of cough during sleep, which initially may be thought to count against a diagnosis of oesophageal cough, may indicate an oesophageal origin. Lower oesophageal sphincter pressure increases physiologically in recumbency preventing reflux in the early stages of the disease. The clues to the diagnosis of cough of oesophageal origin may be obtained by looking for associations between food, eating and cough.

Rhinitis and Postnasal Drip

There is marked geographical variation in the incidence of rhinitis and postnasal drip in the reported series of patients presenting to cough clinics. Patients in the Americas present with symptoms of postnasal drip in up to 50% of cases, whereas rhinitis is reported in approximately 10% in most European experience. The difference for this may be in part societal in that patients from North America are far more likely to describe upper respiratory tract symptoms as postnasal drip. In addition, the diagnosis of postnasal drip or rhinitis is frequently accepted because of a response to ‘specific therapy’ with broad-spectrum, centrally acting antihistamines and systemic decongestants. Such therapy may act in upper airway disease and in asthma. Centrally acting antihistamines may work either on the central pathways of the cough or through a sedating mechanism unrelated to the anatomical site of cough generation.

Until such problems in the definition of postnasal drip and its subsequent specific diagnosis are resolved, rhinitis or rhinosinusitis is probably the preferred term describing this syndrome.

Cough in Cancer Patients

As reviewed by Ahmedazai and Ahmed (Chung, J G et al. 2003) in the cancer patient, who is usually already burdened by several physical and psychological symptoms, cough can become a major source of distress. The cancers that are most commonly associated with cough are, those arising from the airways, lungs, pleura and other mediastinal structures. However, cancers from many other primary sites can metastasize to the thorax and produce the same symptoms.

At presentation, cough is one of the commonest symptoms of lung cancer. Cumulative experience of 650 patients entering the UK Medical Research Centre's multicentre lung cancer trials shows that, overall, cough was the fourth commonest symptom reported at presentation. The actual frequency of cough was 80% in small cell lung cancer (SCLC) and in 70% of non-small cell lung cancer (NSCLC).

Unfortunately, cough is a common consequence of many of the treatments which are used against cancer itself. Studies of long-term survivors of cancer have reported cough as one of the symptoms which both children and adults suffer long after the disease has been treated. The Childhood Cancer Survivor Study which investigated 12390 ex-patients in the USA 5 years or more after their illness found that, compared with siblings, survivors had significantly increased relative risk of chronic cough as well as recurrent pneumonia, lung fibrosis, pleurisy and exercise-induced breathlessness. The propensity for these anticancer therapies to cause pulmonary damage has been known for a long time, although cyclophosphamide-induced lung damage is relatively rare.

The Role of the Vagus Nerve in the Cough Reflex

The vagi are the 10th cranial nerves. They are major nerve trunks comprising of both afferent (sensory) and efferent (motor) neurons. Right and left vagus nerves descend from the cranial vault through the jugular foramina, penetrating the carotid sheath between the internal and external carotid arteries, then passing posterolateral to the common carotid artery. The cell bodies of visceral afferent fibers of the vagus nerve are located bilaterally in the inferior ganglion of the vagus nerve (nodose ganglia). The right vagus nerve gives rise to the right recurrent laryngeal nerve, which hooks around the right subclavian artery and ascends into the neck between the trachea and esophagus. The right vagus then crosses anteriorly to the right subclavian artery and runs posterior to the superior vena cava and descends posterior to the right main bronchus and contributes to cardiac, pulmonary, and esophageal plexuses. It forms the posterior vagal trunk at the lower part of the esophagus and enters the diaphragm through the esophageal hiatus.

The left vagus nerve enters the thorax between left common carotid artery and left subclavian artery and descends on the aortic arch. It gives rise to the left recurrent laryngeal nerve, which hooks around the aortic arch to the left of the ligamentum arteriosum and ascends between the trachea and esophagus. The left vagus further gives off thoracic cardiac branches, breaks up into pulmonary plexus, continues into the esophageal plexus, and enters the abdomen as the anterior vagal trunk in the esophageal hiatus of the diaphragm.

The vagus nerve supplies motor parasympathetic fibers to all the organs except the suprarenal (adrenal) glands, from the neck down to the second segment of the transverse colon.

Whether normal or pathological, cough is a reflex response to increased sensory input from the airways. Sensors within the airways detect irritants, mucus accumulation or inappropriate stretching within the lungs and initiate signals delivered to the brain via sensory (afferent) neurons. These pulmonary afferent neurons are predominantly either C-fibers or A-gamma fibers and travel within the recurrent laryngeal nerve that join the vagi.

The anatomy of the vagus and the physiology of the cough reflex make the ability to control sensory traffic an obvious target for the control of chronic non-productive cough.

Effect of Warming on Neuronal Activity

Since the 19th century it has been known that changing the temperature of nerves impairs their ability to conduct impulses. Based on biochemical principles it is obvious that cooling of tissues should attenuate any biological system and this is indeed true for nerves. However, as early as 1894 it was demonstrated that warming of the nerves could also inhibit transmission (Howell, W. (1894). “The Effect of Stimulation and of Changes in Temperature upon the Irritability and Conductivity of Nerve-fibres.” J Physiol 16(3-4): 298-318; incorporated by reference herein in its entirety). Later Eve (Eve, F. (1900). “The effect of temperature on the functional activity of the upper cervical ganglion.” J Physiol 26(1-2): 119-124; incorporated by reference herein in its entirety) showed that if the cervical ganglion of the rabbit was not held at its upper limit (50 degrees C.) for too long its activity would recover upon cooling. Over the 20th century a number of other researchers showed that heat could inhibit nerve conduction leading to Letcher and Godring (Letcher, F. and S. Goldring (1968). “The effect of radiofrequency current and heat on peripheral nerve action potential in the cat.” J Neurosurg. 29(1): 42-47; incorporated by reference herein in its entirety) to conclude that “The studies suggest the possibility of using heat to modify nerves (in chronic animals for physiologic studies, and in certain pain problems) so that they have no fibers that transmit pain”. However they make no mention of any recovery of the function and the re-establishment of pain sensation upon cooling of those nerves to their original body temperature. However 4 years earlier Brodkey and colleagues (Brodkey, J., Y. Miyazaki, et al. (1964). “Reversible heat lesions with radiofrequency current. A method of stereotactic localization.” J Neurosurg 21(49-53); incorporated by reference herein in its entirety) had shown that carefully controlled radiofrequency current could produce localized small increments in temperature about the tip of the stereotactic electrode in the brain of the cat. In this way, localized temporary blocks of nervous activity could be obtained confirming the final position of an electrode before a permanent lesion is made in the brain. That these small increments in heat produce temporary blocks only, and lead to no permanent destruction of nervous tissue and complete reversible nature was novel. However, they make no mention of this effect in peripheral nerves and conclude that this is a valuable tool for precise location of an electrode before making a permanent brain lesion. In 1973 Rasminsky (Rasminsky, M. (1973). “The effects of temperature on conduction in demyelinated single nerve fibers.” Arch Neurol 28(5): 287-292; incorporated by reference herein in its entirety) described reversible conduction block of demyelinated rat ventral root fibers. He concluded from his observations that the increased susceptibility of demyelinated nerve fibers to heat accounted for the increased susceptibility to heat of patients suffering from multiple sclerosis. In a series of studies on the sciatic nerve branches and the spinal nerve roots of rats Eliasson et al. (Eliasson, S., W. Monafo, et al. (1986). “Differential effects of in vitro heating on rat sciatic nerve branches and spinal nerve roots.” Exp Neurol 93: 57-66; incorporated by reference herein in its entirety) showed that there was selectivity for the inhibitory effects of heat on nerves. They demonstrated that sensory fibers were more heat-sensitive than motor fibers. The concept of a differential temperature sensitivity in motor vs. sensory fibers was not new, although it had been studied previously only with respect to lowering temperature and not to elevating it. This observation provides a basis for this invention that by the application of a controlled amount of heat to the vagi selective and temporary blockade of sensory neurons could be attained. This selective/reversible block could be used to restrict the afferent traffic to the brain to control cough to such extent needed by the patient as to be able to elicit productive cough to eliminate mucus etc. from the airways when necessary but be able to block non-productive cough.

Teaching against this idea are two articles published by Lee and his colleagues. In 2005, (Ruan, T., Q. Gu, et al. (2005). “Hyperthermia increases sensitivity of pulmonary C-fibre afferents in rats.” The Journal of Physiology 565(1): 295-308; incorporated by reference herein in its entirety) published that increasing itrathoracic temperature in an anesthetized rat increased the sensitivity of C-fiber afferents. They summarized their findings thus: “This study was carried out to investigate whether an increase in tissue temperature alters the excitability of vagal pulmonary C-fibres. Single-unit afferent activities of 88 C-fibres were recorded in anaesthetized and artificially ventilated rats when the intrathoracic temperature (T(it)) was maintained at three different levels by isolated perfusion of the thoracic chamber with saline: control (C: approximately 36 degrees C.), medium (M: approximately 38.5 degrees C.) and high (H: approximately 41 degrees C.), each for 3 min with 30 min recovery. Our results showed: (1) The baseline fibre activity (FA) of pulmonary C-fibres did not change significantly at M, but increased drastically (>5-fold) at H. (2) The C-fibre response to right-atrial injection of capsaicin (0.5 microg kg(−1)) was markedly elevated at H (deltaFA=5.94+/−1.65 impulses s(−1) at C and 13.13+/−2.98 impulses s(−1) at H; P<0.05), but not at M. Similar increases in the C-fibre responses to other chemical stimulants (e.g. adenosine, etc.) were found at H; all the enhanced responses returned to control in 30 min. (3) The C-fibre response to lung inflation was also significantly potentiated at H. In sharp contrast, there was no detectable change in either the baseline activity or the responses to lung inflation and deflation in 10 rapidly adapting pulmonary receptors and 10 slowly adapting pulmonary receptors at either M or H. (4) The enhanced C-fibre sensitivity was not altered by pretreatment with indomethacin or capsazepine, a selective antagonist of the transient receptor potential vanilloid type 1 (TRPV1) receptor, but was significantly attenuated by ruthenium red that is known to be an effective blocker of all TRPV channels. (5) The response of pulmonary C-fibres to a progressive increase in T(it) in a ramp pattern further showed that baseline FA started to increase when T(it) exceeded 39.2 degrees C. In conclusion, a pronounced increase in the baseline activity and excitability of pulmonary C-fibres is induced by intrathoracic hyperthermia, and this enhanced sensitivity probably involves activation of temperature-sensitive ion channel(s), presumably one or more of the TRPV receptors, expressed on the C-fibre endings.” These finding strongly suggest that any significant increase in body temperature up to 41° C. would increase the airway sensitivity and enhance the cough reflex.

One year later Lee and his colleagues (Ni, D., Q. Gu, et al. (2006). “Thermal sensitivity of isolated vagal pulmonary sensory neurons: role of transient receptor potential vanilloid receptors.” Am J Physiol Regul Integr Comp Physiol 29(3): R541-550; incorporated by reference herein in its entirety) followed up on their earlier findings and used patch-clamp electrophysiology techniques to show that increasing the temperature of isolated vagal pulmonary sensory neurons increased their sensitivity. They reported that “On the basis of these results, we conclude that increasing temperature within the normal physiological range can exert a direct stimulatory effect on pulmonary sensory neurons, and this effect is mediated through the activation of TRPV1, as well as other subtypes of TRPV channels.” However, in both studies, Lee and colleagues did not raise the temperature of their preparations beyond 41 degrees C. Still higher temperatures may be needed to inhibit these neurons, however, this is not addressed by the authors.

Both of these studies would lead one to conclude that, whereas previous publications may describe an inhibitory effect of increased temperature, when it comes to the pulmonary afferents that transmit information from the airways to the brain, that increasing temperature would do the opposite and lead to increased sensitivity and probably result in lower thresholds for the cough reflex.

Heating of nerve axons can also give rise to permanent destruction of the nerve. This has been the basis of various therapeutic approaches, for example the ablation of renal nerves for the treatment of hypertension (Investigators, S. H.-., M. Esler, et al. (2010). “Renal sympathetic denervation in patients with treatment-resistant hypertension (The Symplicity HTN-2 Trial): a randomised controlled trial.” Lancet 376(9756): 1903-1909.; Esler, M. D., H. Krum, et al. (2012). “Renal Sympathetic Denervation for Treatment of Drug-Resistant Hypertension: One-Year Results From the Symplicity HTN-2 Randomized, Controlled Trial.” Circulation 126(25): 2976-2982.; Bohm, M., D. Linz, et al. (2013). “Renal sympathetic denervation: applications in hypertension and beyond.” Nature Reviews Cardiology 10: 465-76; each of which is incorporated by reference herein in its entirety). The reversibility of the heating effect on nerves depends greatly on the temperature that the nerves are heated to. It was demonstrated that in the dog phrenic nerve permanent nerve injury occurred at temperatures of 51±6 degrees C. (median 49 degrees C., range: 45-65 degrees C.), which was significantly higher than the temperature at which transient inhibition of the nerve occurred (47±3 degrees C.) (Bunch, T. J., G. K. Bruce, et al. (2005). “Mechanisms of Phrenic Nerve Injury During Radiofrequency Ablation at the Pulmonary Vein Orifice.” Journal of Cardiovascular Electrophysiology 16(12): 1318-1325; incorporated by reference herein in its entirety). These data would suggest that there is a significant margin of safety between the temperatures needed for transient nerve inhibition and permanent ablation and an approximately 14 degrees C. (37-51 degrees C.) working range within which to optimize the transient inhibition of vagal afferent nerve fibers for the treatment of cough.

The observations described above open up the possibility of using heat in a number of ways to inhibit the cough reflex. Firstly, heat can be applied to the vagus nerves in the neck. This would have to be enough heat to transiently inhibit nerve transmission but not enough to cause permanent ablation. Using the data from Burch et al (Bunch, Bruce et al. 2005) these temperatures would be in the region of 47±3° C. Similarly, the same type of transient nerve block using heat in the region of 47±3° C. could be applied to the recurrent laryngeal nerves between where they exit the bronchus and where they join the vagus in the chest. Alternatively, the same amount heat (47±3 degrees C.) could be applied directly to the trachea so as to temporarily inhibit the afferent nerve endings within the trachea thus inhibiting the cough reflex. Since the afferent nerves in the trachea run from the cranial end towards the carina, and eventually form the recurrent laryngeal nerves, the heat should be applied to the cranial end of the trachea, preferentially to the first 7-10 tracheal rings, as this is where most of the afferent nerve endings are situated (Baluk, P. and G. Gabella (1991). “Afferent nerve endings in the tracheal muscle of guinea-pigs and rats.” Anat Embryol 183: 81-87; incorporated by reference herein in its entirety) and application of heat at this end of the trachea should not interfere with sensory neurons that arise closer to the carina thus allowing the subject to have intact afferent innervation to part of the trachea. Alternatively, a greater amount of heat sufficient to permanently ablate the afferent nerves but not damage the tracheal tissue could be applied to the trachea, in particular to the cranial end of the trachea and especially the first 7-10 tracheal rings. The level of heat necessary for this application would have to be higher than that needed for temporary inhibition and in the region of the parameters described by Bunch et al, of 51±6 degrees C. (median 49 degrees C., range: 45-65 degrees C.) (Bunch, Bruce et al. 2005). This heat could be applied using various devices and methodologies including direct heat from a heat source such as a cuff, heating plate or probe applied to the outside or the lumen of the trachea for such time necessary to cause the permanent ablation of the cough response when that region of the trachea is stimulated. Heat could also be applied to the trachea using other methodologies such as radiofrequency or ultrasound. These methodologies could be “tuned” to apply either enough heat so as to bring about either temporary inhibition of the afferent nerves resulting in prevention of cough, in the region of 47±3 degrees C., or higher levels of heat so as to cause permanent ablation of the nerves, in the region of 51±6 degrees C. These methodologies would be similar in nature and outcome as far as nerve ablation as those previously described for renal nerve ablation for the treatment of hypertension (Investigators, Esler et al. 2010; Esler, Krum et al. 2012; Bohm, Linz et al. 2013).

There is a need for better systems and methods for treating cough. Various configurations are described herein, wherein heat may be utilized to control the pulmonary afferents to inhibit cough.

### BRIEF DESCRIPTION OF THE DRAWINGS

FIG. 1 illustrates various aspects of a process wherein a chronic cough patient may be treated using thermodynamic neuromodulation.

FIG. 2 illustrates various aspects of a system for treating a patient using thermodynamic neuromodulation.

FIGS. 3-8B illustrate various aspects of components which may be utilized in systems for treating patients using thermodynamic neuromodulation.

FIGS. 9A-9B illustrate various aspects of systems for treating patients using thermodynamic neuromodulation.

FIGS. 10-18B illustrate various aspects of components which may be utilized in systems for treating patients using thermodynamic neuromodulation.

FIGS. 19A-19B illustrate various aspects of a system for treating a patient using thermodynamic neuromodulation.

FIG. 20 illustrates a chart reading featuring experimental confirmation data pertinent to a cough study.

### SUMMARY

Hypersensitivity of the tissues or inappropriate responses to non-noxious stimuli within the trachea and bronchi result in excessive afferent traffic from the upper airways leads to a non-productive chronic cough.

One embodiment provides cuffs surgically placed around the vagus nerves that comprise of heating elements that could be a wire of known resistance that when a current is passed through it generates heat. The heating elements within the cuffs may be connected via wire to a “control module”. The “control module” may comprise a battery, circuitry for controlling the current supplied to the cuffs and a switch to activate the control module. The switch may be activated by a second “key unit” configured to transmit a signal through the skin to the “control module”. Integrated into the cuff may be a means of measuring the temperature within the cuff which may comprise a thermocouple. This thermocouple may provide feedback to the “control unit” to maintain a set temperature. When the patient wishes to inhibit cough, in one embodiment they may place the “key unit” against the skin over the area of the implanted “control module”, thus activating the “control module”. The temperatures used for the inhibition of cough may be between 40 degrees C. and 50 degrees C., and preferably between 43 degrees C. and 48 degrees C. The final temperature settings may vary between individuals depending on the sensitivity of the nerves to heat and the placement of the cuffs during surgery.

In another embodiment a cuff arrangement may be complemented with a “control module” comprising circuitry to control the current to the one or more cuffs, and a means for receiving electrical power from an outside source by means of an antenna. The “key unit” that is outside of the body may house a battery, an antenna, and a means of transmitting power to the control module. When the patient wishes to inhibit cough they may place the “Key unit” against the skin over the area of the implanted “control module”, thus activating the “control module”.

Another embodiment is directed to a system for managing unproductive cough in a patient, comprising: an applicator comprising a resistive heating element and being configured to be positioned adjacent a portion of a targeted nerve tissue for treatment; a power source configured to provide electrical current to the resistive heating element; and a current controller operatively coupled to the power source and configured to raise the temperature of the portion of the targeted nerve tissue to inhibit nerve conduction. The targeted nerve tissue may comprise at least one vagal afferent nerve. The resistive heating element may be configured to be positioned immediately adjacent to the at least one vagal afferent nerve. The resistive heating element may be configured to at least partially surround the at least one vagal afferent nerve. The applicator further may comprise a temperature sensor operatively coupled to the current controller. The temperature sensor may be configured to produce an electrical signal representative of a nearby temperature and deliver the electrical signal to the current controller, the current controller being configured to vary the electrical current provided to the resistive heating element based at least in part upon the electrical signal from the temperature sensor. The current controller may be configured to maintain the temperature of the targeted nerve tissue portion within a desired range for a period of time. The temperature sensor may comprise a sensor selected from the group consisting of: a bimetallic sensor or switch, a fluid expansion sensor or switch, a thermocouple, a thermistor, a Resistance Temperature Detector, and an infrared pyrometer. The desired range may be between about 38 degrees Celsius and about 46 degrees Celsius. The desired range may be within ±2 degrees Celsius of a nominal temperature within a range of about 38 degrees Celsius and about 46 degrees Celsius. The applicator further may comprise an electrical activity sensor operatively coupled to the current controller and configured to produce an electrical signal representative of electrical activity of at least one nerve. The controller may be configured to interpret the signal from the electrical activity sensor and vary the current to the resistive heating element at least in part relative to the electrical signal representative of electrical activity of the at least one nerve. The controller may be configured to maintain a level of activity of the targeted nerve tissue portion within a desired range for a period of time. The controller may be operatively coupled to a temperature sensor and is configured to also maintain a temperature of the targeted nerve tissue portion within a desired range for a period of time. The current controller may be further configured to deliver the electrical current in a pulsatile fashion. The pulsatile fashion may comprise current pulses delivered have a time duration between about 1 millisecond and about 100 seconds. The pulsatile fashion may comprise a current pulse duty cycle of between about 99% and 0.1%. The current controller further may be configured to be controlled for an output characteristic selected from the group consisting of: current amplitude, pulse duration, duty cycle, and overall energy delivered. The current controller may be configured to be responsive to at least one patient input. The current controller may be configured such that the at least one patient input triggers a delivery of current to the resistive heating element. The applicator may be placed to at least 60% circumferentially surround a vagal afferent nerve or vagal afferent nerve bundle.

Another embodiment is directed to a method for managing unproductive cough in a patient, comprising: providing an applicator comprising a resistive heating element and being configured to be positioned adjacent a portion of a targeted nerve tissue for treatment; providing a power source configured to provide electrical current to the resistive heating element; providing a current controller operatively coupled to the power source and configured to raise the temperature of the portion of the targeted nerve tissue to inhibit nerve conduction; and modulating the temperature of the portion of the targeted nerve tissue to inhibit nerve conduction. The targeted nerve tissue may comprise at least one vagal afferent nerve. The resistive heating element may be configured to be positioned immediately adjacent to the at least one vagal afferent nerve. The resistive heating element may be configured to at least partially surround the at least one vagal afferent nerve. The applicator further may comprise a temperature sensor operatively coupled to the current controller. The temperature sensor may be configured to produce an electrical signal representative of a nearby temperature and deliver the electrical signal to the current controller, the current controller being configured to vary the electrical current provided to the resistive heating element based at least in part upon the electrical signal from the temperature sensor. The method further may comprise utilizing the current controller to maintain the temperature of the targeted nerve tissue portion within a desired range for a period of time. The temperature sensor may comprise a sensor selected from the group consisting of: a bimetallic sensor or switch, a fluid expansion sensor or switch, a thermocouple, a thermistor, a Resistance Temperature Detector, and an infrared pyrometer. The desired range may be between about 38 degrees Celsius and about 46 degrees Celsius. The desired range may be within ±2 degrees Celsius of a nominal temperature within a range of about 38 degrees Celsius and about 46 degrees Celsius. The applicator further may comprise an electrical activity sensor operatively coupled to the current controller and configured to produce an electrical signal representative of electrical activity of at least one nerve. The controller may be configured to interpret the signal from the electrical activity sensor and vary the current to the resistive heating element at least in part relative to the electrical signal representative of electrical activity of the at least one nerve. The method further may comprise utilizing the current controller to maintain a level of activity of the targeted nerve tissue portion within a desired range for a period of time. The controller may be operatively coupled to a temperature sensor, the method further comprising utilizing the current controller to also maintain a temperature of the targeted nerve tissue portion within a desired range for a period of time. The current controller may be further configured to deliver the electrical current in a pulsatile fashion. The pulsatile fashion may comprise current pulses delivered have a time duration between about 1 millisecond and about 100 seconds. The pulsatile fashion may comprise a current pulse duty cycle of between about 99% and 0.1%. The current controller further may be configured to be controlled for an output characteristic selected from the group consisting of: current amplitude, pulse duration, duty cycle, and overall energy delivered. The current controller may be configured to be responsive to at least one patient input. The current controller may be configured such that the at least one patient input triggers a delivery of current to the resistive heating element. The applicator may be placed to at least 60% circumferentially surround a vagal afferent nerve or vagal afferent nerve bundle.

### DETAILED DESCRIPTION

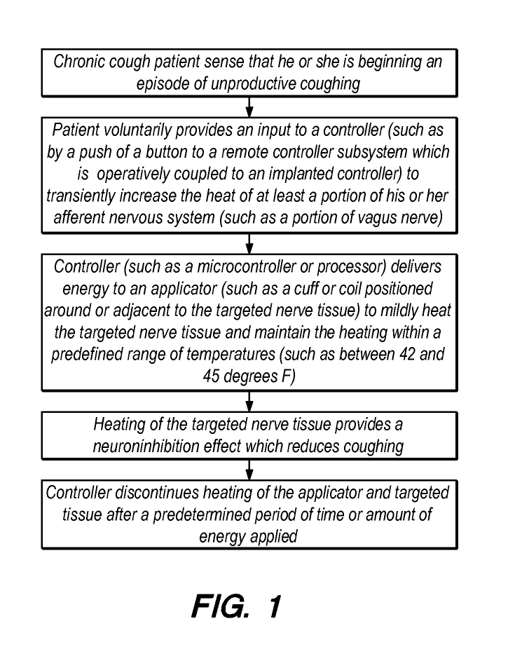

Referring to FIG. 1, in one embodiment, a chronic cough patient may sense that he or she is beginning an episode of unproductive cough. The patient may voluntarily provide an input to a controller, such as by a push of a button on a remote controller subsystem which is operatively coupled to an implantable controller, to transiently increase the heat of at least a portion of his or her afferent nervous system, such as a portion of a vagus nerve. The controller (such as a microcontroller or processor) may be configured to deliver energy to an applicator (such as a cuff or coil positioned around or adjacent to the targeted nerve tissue) to mildly heat the targeted nerve tissue and maintain the heating within a predefined range of temperatures (such as between about 42 and about 45 degrees F.). Heating of the targeted nerve tissue provides a neuroinhibition effect which reduces coughing in the patient. After a predetermined period of time or amount of power applied, such as by way of non-limiting example, 10-100 sec and 100 to 2000 mW, the controller may be configured to discontinue or decrease the heating of the targeted tissue.

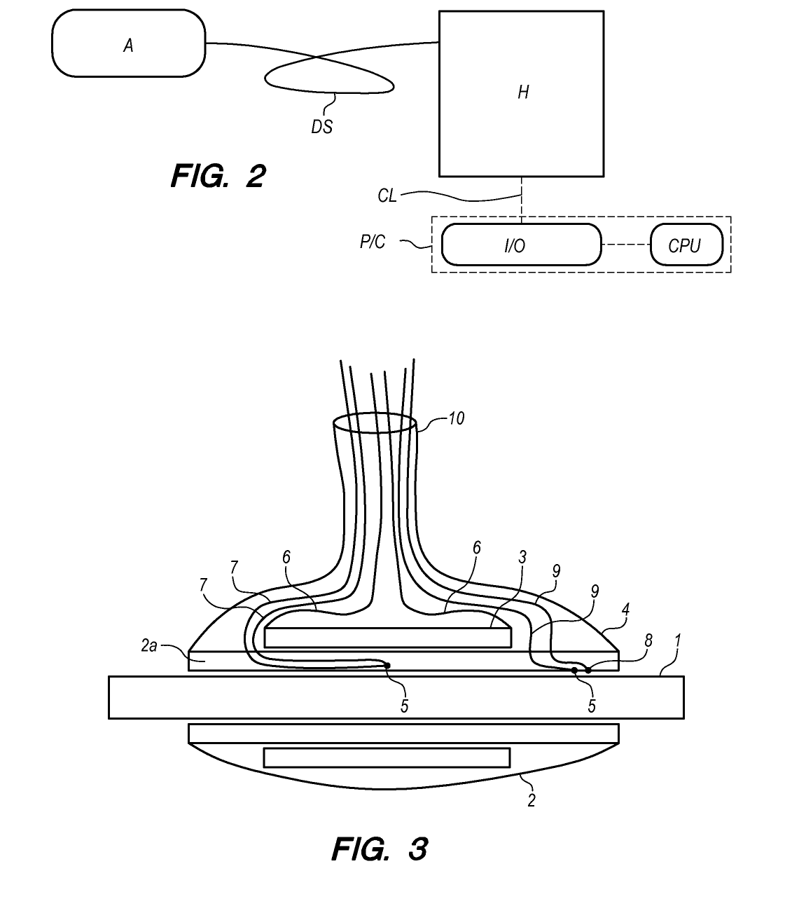

Referring to FIG. 2, a suitable heat delivery system comprises one or more applicators (A) configured to provide heat output to the targeted tissue structures. The heat may be generated within the applicator (A) structure itself. The one or more delivery segments (DS) serve to transport, or guide, electricity to the applicator (A). In an embodiment wherein the heat is generated within the applicator (A), the delivery segment (DS) may simply comprise an electrical connector to provide power to the heat source and/or other components which may be located distal to, or remote from, the housing (H). The one or more housings (H) preferably are configured to serve power to the heat source and operate other electronic circuitry, including, for example, telemetry, communication, control and charging subsystems. External programmer and/or controller (P/C) devices may be configured to be operatively coupled to the housing (H) from outside of the patient via a communications link (CL), which may be configured to facilitate wireless communication or telemetry, such as via transcutaneous inductive coil configurations, between the programmer and/or controller (P/C) devices and the housing (H). The programmer and/or controller (P/C) devices may comprise input/output (I/O) hardware and software, memory, programming interfaces, and the like, and may be at least partially operated by a microcontroller or processor (CPU), which may be housed within a personal computing system which may be a stand-alone system, or be configured to be operatively coupled to other computing or storage systems. In a further embodiment, the applicator may contain a temperature sensor, such as a resistance temperature detector (RTD), thermocouple, or thermistor, etc. to provide feedback to the processor in the housing to assure that the tissue temperature is controlled, as is discussed in further detail herein.

The Applicator A may consist of a polymer tube that effectively surrounds the target tissue, such as a nerve. This tube may further be configured to be surrounded by a flexible resistive heating element, which may be, in turn surrounded by another layer of polymer that may serve as an insulating layer to reduce the rate of dissipation of the heat to the surrounding tissue.

The Applicator may be made to fit the target tissue snugly in order to more directly and efficiently deliver heat. By way of non-limiting example, a nerve cuff may be configured to provide an inner diameter that surrounds the Vagus nerve of a patient, and is between 80-150% of the effective diameter of the target nerve.

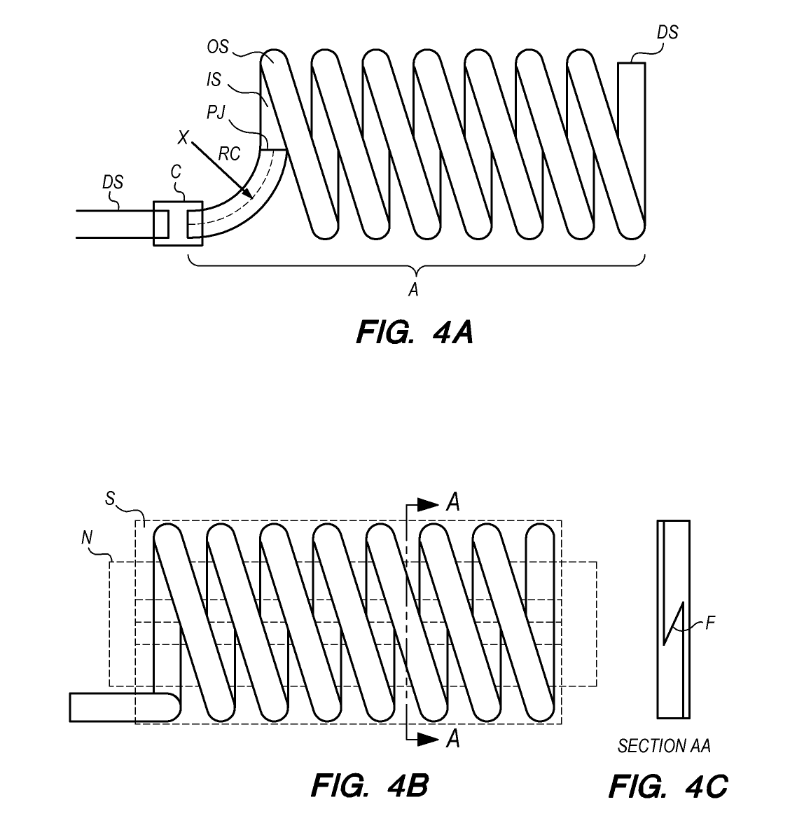

FIG. 3 depicts an embodiment of the present invention, wherein the Target Tissue 1 is surrounded by a Applicator A that forms a heater cuff Applicator 2. The Cuff forms a tube with inner diameter as close as possible to the diameter of the nerve without being substantially smaller. The cuff should circumferentially enclose the nerve as completely as possible. The cuff should either be flexible enough to open and allow placement over the nerve, such as if it were made of all elastic polymers with only thin layers of relatively flexible metals or be configured to utilize braided cables composed of thin wire for the heating element(s), or have some means of opening to place on nerve, such as with a hinge or a small segment of flexible material. Cuff 2 comprises Inner Layer 2a which is preferably thin, flexible and heat conductive.

A few possible materials for the inner layer are Silicone, Urethane, Polyimide. Specific examples of such low durometer, unrestricted grade implantable materials are MED-4714 or MED4-4420 from NuSil, which have a Shore A durometer of about 16, while that of natural latex is nominally about 25. They also have a thermal conductivity of about 0.82 Wm⁻¹K⁻¹, and a thermal diffusivity, α, of about 0.22 mm²s⁻¹. This is about 50% greater than that of most tissues, which has a thermal diffusivity approximately equal to that of water, α=0.14 mm²s⁻². Surrounding the inner layer may be a Heating Element 3. Heating Element 3 may be configured to be as thin and flexible as possible while maintaining its mechanical and heat production integrity. It could either be flexible or segmented to allow placement on the nerve. At least two electrical connections, shown as Heater Wire 6, may be utilized to power Heating Element 3. If variations in heating pattern are desired, Heating Element 3 may be separated into segments which may be controlled independently. Such segments of Heating Element 3 maybe wired independently or with a common ground (return lead). Heating Element 3 may be made of anything that would convert electricity to heat, the simplest of these materials being resistive metals. Example materials for the heating element are nichrome, kanthal, cupronickel, and Inconel. These metals have the benefit of being relatively flexible. Alternately, a Heating Element may be configured to utilize braided cables composed of thin wires of the abovementioned metals. Alternately, a Heating Element may be produced using a polymer doped with electrically conductive powder or particulates resulting in a flexible conductor, such as Metal Rubber, which is produced by NanoSonics, Inc. Alternately, a polymer-coated metallic resistive heating Element, such as Silicone Rubber Heaters, Polymer Thick Film Heaters, UltraFlex and Kapton Heaters, available from thermo Heating Elements, LLC may be utilized. These heaters are capable of producing between 0.2-10 W/cm², and range in thickness from 25 μm-1.6 mm. Alternately, smaller segments of more rigid ceramic heating elements can be used, such as, but not limited to, molybdenum disulfide, barium titanate and lead titanate. These segments may be electrically connected to each other or to the power source individually with highly conductive wire. Alternately, a single or array of Peltier elements may be used to heat the target tissue. A Peltier device may be configured to move heat from one side to the other when an electrical current is applied, and are capable of 10-15% Carnot efficiencies. This may be used to draw heat from the outside layer of the cuff to the inside of the cuff. This may also be used in reverse to cool the nerve. Alternately, a Peltier device may also be used in conjunction with a heating element to effectively neutralize any heating or cooling that would be applied to the surrounding tissue.

Some heating elements will change in resistance as their temperature changes. If this change is predictable and large enough to be measured effectively this change can be used as feedback in a closed loop control. If the resistance change is not sufficient or predictable then a temperature sensor, such as Temperature Sensor 5 may be configured to monitor the temperature of or adjacent to Target Tissue. Examples of possible temperature sensors are thermocouples, thermistors, and thermopiles. Temperature Sensor 5 would preferably be placed as close to the nerve as possible. Alternately, the system may be configured to monitor temperature in multiple locations within the cuff to be sure that the temperature is consistent over the area to be heated. Alternatively multiple sensors and independent heating elements can be used to provide a desired temperature profile in different areas of the nerve. Temperature sensors may also be placed in the outer layers of the cuff to monitor the temperature of the tissue outside the cuff. Each temperature sensor may be connected to the control unit via two electrically insulated conductive wires, such as Temperature Sensor Wires 7, or in an arrangement with a common return wire. Examples of temperature sensors that may be used include but are not limited to a bimetallic sensor or switch, a fluid expansion sensor or switch, a thermocouple, a thermistor, a Resistance Temperature Detector, and an infrared pyrometer. These may be deployed independently, or in combination. Multiple sensors may be employed, either redundantly, or in combination.

In configurations where bimetallic or fluid expansion switches are used, they may be integrated in to an interlock circuit that carries the therapeutic current, or as binary sensors that indicate that the sensed temperature is above, below, or within the desired range. Dual switch sensors may be deployed in “normally-open” and “normally-closed” pairs, either in series or in parallel, to provide for a sensing range, the overlap of them forming the sensor deadband within which the current is allowed to flow to the resistive heater in the applicator.

In the case of the pyrometer, an optical fiber may be used to conduct the sensed light from the tissue to a detector within the housing of the controller (not shown for simplicity). Such a fiber would need to be transmissive in and around the 10 μm wavelength region, as that corresponds to the blackbody radiation at the temperatures of interest. Chalcochinide glasses and hollow waveguides are well suited to this application. Similarly, the detector must be responsive in the same spectral range noted above. In an alternate configuration, the detector may be placed within Applicator (cuff) and the resultant electrical signals transmitted to the controller.

The Heating Element 3 and Temperature Sensor 7 may be configured to be at least partially encapsulated by Insulation 4 in order to electrically isolate them from tissue and to shield them from direct exposure to body fluids or ingrowth. This material may also serve to hold or reflect the heat back into the nerve so the surrounding tissue is heated as little as possible. Preferably the outer insulating layer of the cuff would have a low thermal conductivity but be as thin as flexible as possible, as was described elsewhere herein. Delivery Segment 10 is equivalent to that described elsewhere herein and in the referenced material as Delivery Segment DS, or Delivery Segments DSx. Likewise, Cuff cuff is equivalent to Applicator A.

Another embodiment may include a single or multiple Electroneurographic (ENG) nerve recoding electrode(s), shown as elements 8 and 9 in FIG. 3, in the Applicator (cuff) to sense and/or measure nerve electrical activity and to sense and/or measure any changes in nerve behavior as a result of the heating. This signal may be used as feedback for the controller. ENG recording is well documented in Methods for neural ensemble recordings by M. Nicolelis (2008, CRC Press, vol. 2), and Implanted Neural Interfaces: Biochallenges and Engineered Solutions, by W. Grille, et al (2009, doi: 10.1146/annurev-bioeng-061008-124927), and Selective Recording of the Canine Hypoglossal Nerve Using a Multicontact Flat Interface Nerve Electrode, by P. Yoo, et al. (2005, doi: 10.1109/TBME.2005.851482), and Neural Prostheses for Restoration of Sensory and Motor Function, edited by J. Chapin, et al (2001, ISBN:978-0-8493-2225-9), which are incorporated herein in their entirety.

In an alternate embodiment, ENG measurements may be made during periods when the heating element is inactive to eliminate it as a source of noise, such as may be the case for alternating current configurations. The delay between such heating and ENG measurement may be substantially instantaneous, and measurements be recorded for as long as 9 ms, 35 ms, and 78 ms after the cessation of energy to the heater and substantially not affect the aggregate tissue temperature of 1 mm, 2 mm, and 3 mm diameter target structures, respectively. This can be appreciated by considering that the e⁻² thermal relaxation time, τᵣ, of a cylinder is approximately equal to d²/16α, where d is the diameter and α is the tissue thermal diffusivity as described above. An ENG electrode may also be made from the conductive polymers, such as Metal Rubber, albeit with a nominally greater conductivity than that of the Heating Elements described herein.

The Applicator may be detachably attached to a control and power module within Housing H via a Delivery Segment DS and connector C. This Delivery Segment may be configured to be as flexible as possible while providing sufficient protection for the wires. The wires may be covered in an insulating protective sleeve. Example materials for constructing the sleeve material are silicone and urethane. In one embodiment the cuff lead may be fabricated to have undulations U to allow for maximum flexibility and to isolate the distal end of the nerve cuff from any movement along the length of the cuff lead, as described elsewhere herein.

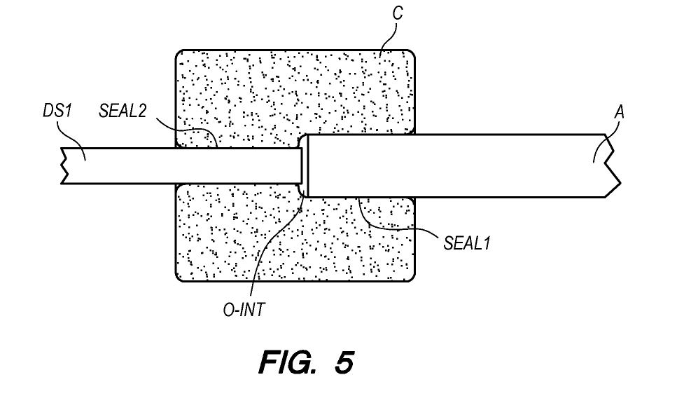

As described herein, applicators suitable for use with the present invention may be configured in a variety of ways. Referring to FIGS. 4A-4C, a helical applicator with a spring-like geometry is depicted. Such a configuration may be configured to readily bend with, and/or conform to, a targeted tissue structure (N), such as a nerve, nerve bundle, vessel, or other structure to which it is temporarily or permanently coupled. Such a configuration may be coupled to such targeted tissue structure (N) by “screwing” the structure onto the target, or onto one or more tissue structures which surround or are coupled to the target. As shown in the embodiment of FIG. 4A, an electrical cable may be connected to, or be a contiguous part of, a delivery segment (DS), and separable from the applicator (A) in that it may be connected to the applicator via connector (C). Alternately, it may be affixed to the applicator portion without a connector and not removable. Both of these embodiments are also described with respect to the surgical procedure described herein. Connector (C) may be configured to serve as a slip-fit sleeve into which both the distal end of Delivery Segment (DS) and the proximal end of the applicator are inserted. The term electrical cable is used herein to describe an electrical wire, or plurality of wires that may be used to convey electrical power and/or signals to and from the applicator and/or housing.

FIG. 5 shows an exemplary embodiment, wherein Connector C may comprise a single flexible component made of a polymer material to allow it to fit snugly over the substantially round cross-sectional Delivery Segment DS1, and Applicator A. These may be electrical leads such as electric cables and/or wires, and similar mating structures on the applicator, and/or delivery segment, and/or housing to create a substantially water-tight seal, shown as SEAL1 & SEAL2, that substantially prevents cells, tissues, fluids, and/or other biological materials from entering the Electrical Interface O-INT.

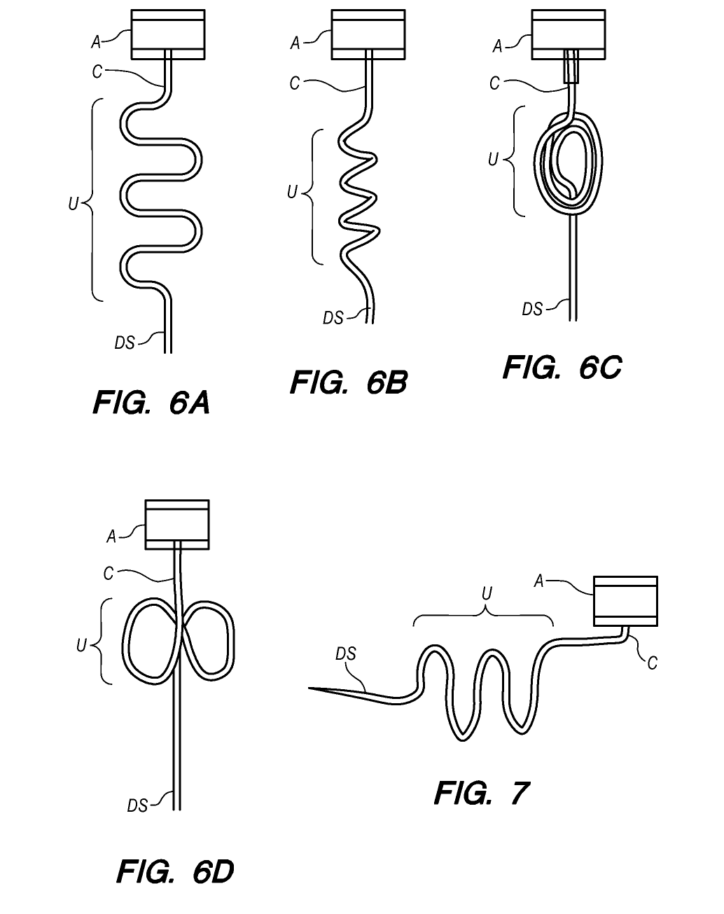

A Delivery Segment (DS) may also be configured to include Undulations (U) in order to accommodate possible motion and/or stretching/constricting of the target tissues, or the tissues surrounding the target tissues, and minimize the mechanical load (or “strain”) transmitted to the applicator from the delivery segment and vice versa. Undulations (U) may be pulled straight during tissue extension and/or stretching. Alternately, Undulations (U) may be integral to the applicators itself, or it may be a part of the Delivery Segments (DS) supplying the applicator (A). The Undulations (U) may be configured of a succession of waves, or bends in the waveguide, or be coils, or other such shapes. FIG. 6A-6D illustrate a few of these different configurations in which Undulations U are configured to create a strain relief section of Delivery Segment DS prior to its connection to Applicator A via Connector C. FIG. 6A illustrates a Serpentine section of Undulations U for creating a strain relief section within Delivery Segment DS and/or Applicator A. FIG. 6B illustrates a helical section of Undulations U for creating a strain relief section within Delivery Segment DS and/or Applicator A. FIG. 6C illustrates a Spiral section of Undulations U for creating a strain relief section within Delivery Segment DS and/or Applicator A. FIG. 6D illustrates a Bowtie section of Undulations U for creating a strain relief section within Delivery Segment DS and/or Applicator A. Target Tissue resides within Applicator in these exemplary embodiments, but other configurations, as have been described elsewhere herein, are also within the scope of the present invention.

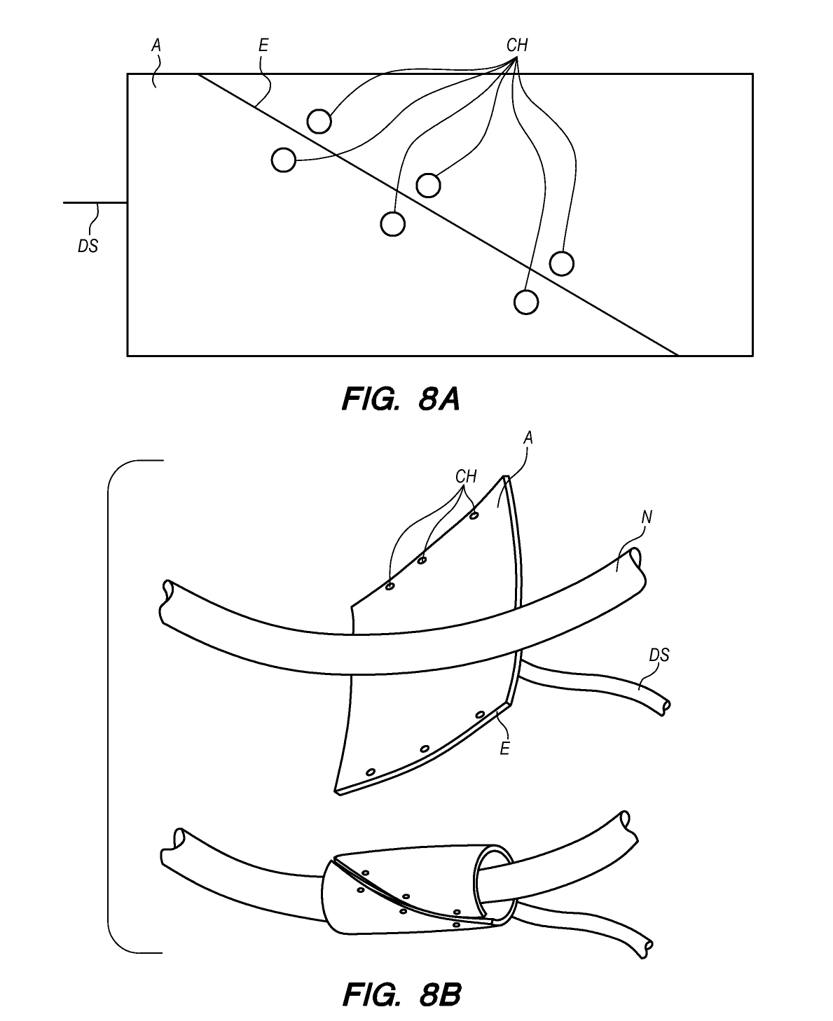

FIG. 7 shows an alternate embodiment, wherein Applicator A may be configured such that it is oriented at an angle relative the Delivery Segment DS, and not normal to it as was illustrated in the earlier exemplary embodiments. Such an angle might be required, for example, in order to accommodate anatomical limitations, such as the target tissue residing in a crevice or pocket, as may the case for certain peripheral nerves.

Alternately, DS containing Undulations (U) may be enclosed in a protective sheath or jacket to allow DS to stretch and contract without encountering tissue directly.

A rectangular slab applicator may be configured to be like that of the aforementioned helical-type, or it can have a permanent Delivery Segment (DS) attached/inlaid. For example, a slab may be formed such that is a limiting case of a helical-type applicator, such as is described elsewhere herein, for explanatory purposes, and to make the statement that the attributes and certain details of the aforementioned helical-type applicators are suitable for this slab-like as well and need not be repeated.

In the embodiment depicted in FIGS. 8A-8B, Applicator (A) is fed by Delivery Segment (DS) and the effectively half-pitch helix is closed along the depicted edge (E), with closure holes (CH) provided, but not required, to surround target tissue N.

It should also be understood that the helical-type applicator described herein may also be utilized as a straight applicator, such as may be used to provide heat along a linear structure like a nerve, etc.

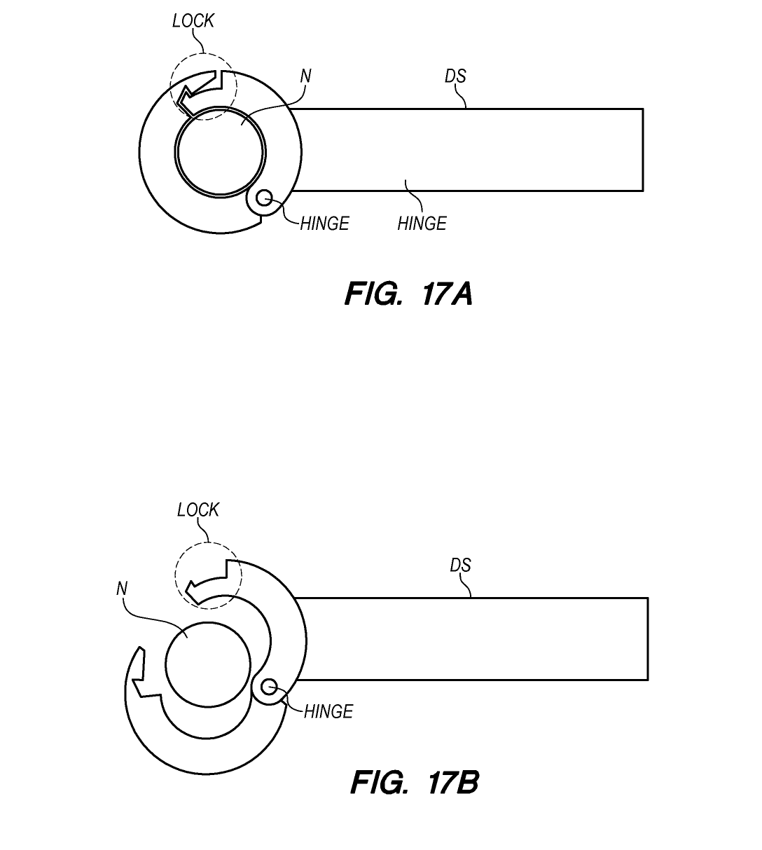

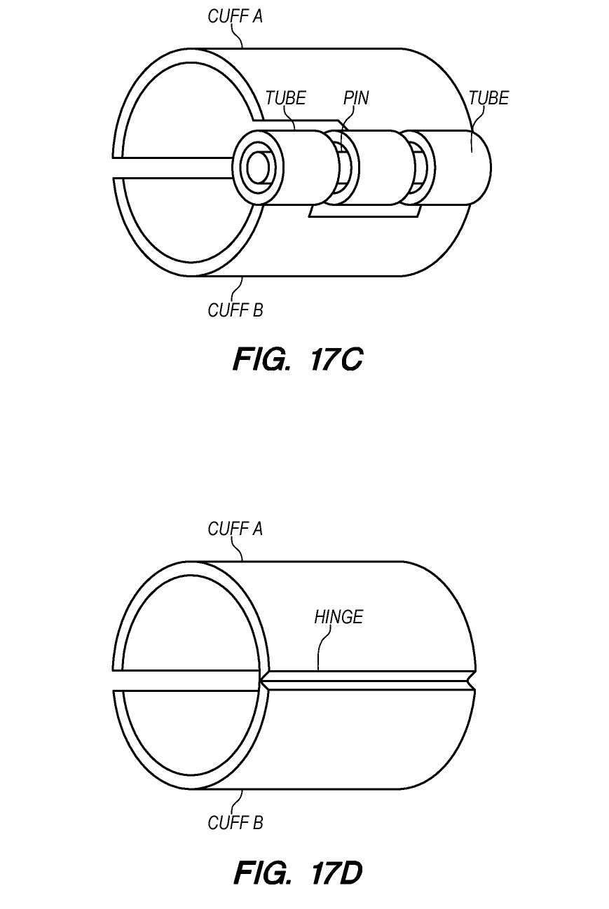

The embodiment of FIGS. 17A-17B, is similar to those of FIGS. 3 and 8A-8B, with the additions of a hinge and a locking feature. Hinge [HINGE] is shown in the open position as applicator A is placed about target N.

Referring to FIG. 17C, the hinge [HINGE] may be constructed from a pin [PIN] attached to one side of the cuff [CUFF A] that is rotatably coupled to a split tube [TUBE] attached to the other side of the cuff [CUFF B].

Alternately, referring to FIG. 17D, the configuration may comprise a living hinge, or a small flexible section [HINGE] of the cuff between two more rigid sections [CUFF A] and [CUFF B]. FIGS. 17A-17B also show the device in place and secured about target N utilizing Locking Mechanism [LOCK]. Locking Mechanism [LOCK] may be any geometry that resists opening once the cuff is closed around the nerve. This can be a hook like geometry that relies on a small amount of flexibility in the cuff structure to bend out of shape while closing or opening as shown in FIGS. 17A-17B. Alternately it could require the operator to move a secondary piece of material that would prevent the opening of the cuff when not desired.

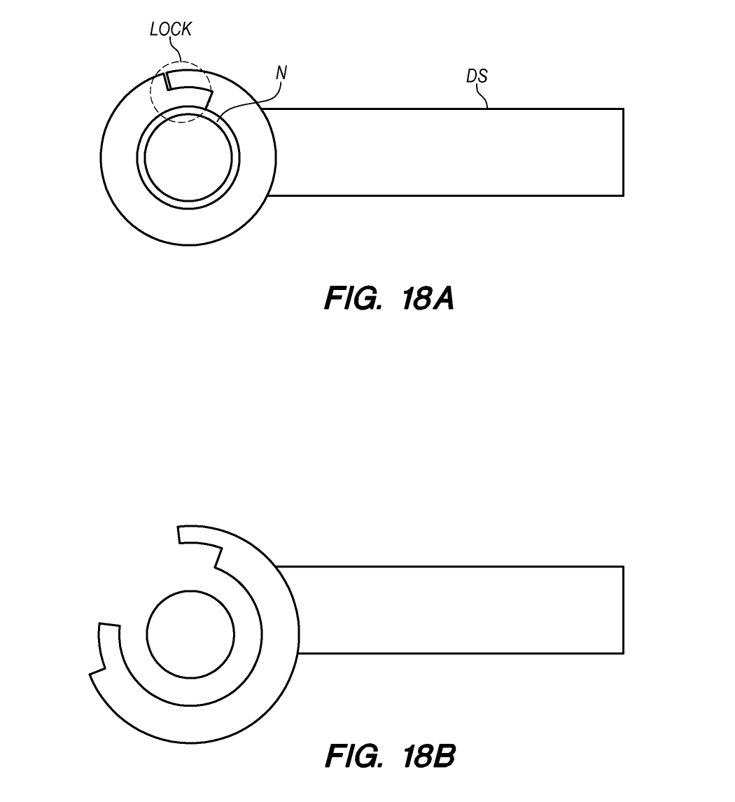

The embodiment of FIGS. 18A-18B is similar to that of FIGS. 17A-17B, with the addition of a construction with flexibility that is adequate to apply over the nerve being used in lieu of the hinge mechanism.

As described herein, an Applicator is placed at, or adjacent, or nearby a neural target. In certain embodiments, a cuff is used to engage the target. A cuff need not completely surround a nerve. It may surround as little as 60% and still create a reliable fit in most instances.

Changes to the output of the heat source may be made to, for example, the output power, exposure duration, exposure interval, duty cycle, pulsing scheme, temperature, energy delivered, etc. It is to be understood that the term “constant” does not simply imply that there is no change in the signal or its level, but maintaining its level within an allowed tolerance. Such a tolerance may be of the order of ±20% on average. However, patient and other idiosyncrasies may also be need to be accounted and the tolerance band adjusted on a per patient basis where a primary and/or secondary therapeutic outcome and/or effect is monitored to ascertain acceptable tolerance band limits. As mentioned elsewhere herein, a control band of ±2° C. may be sufficient to produce reliable therapy.

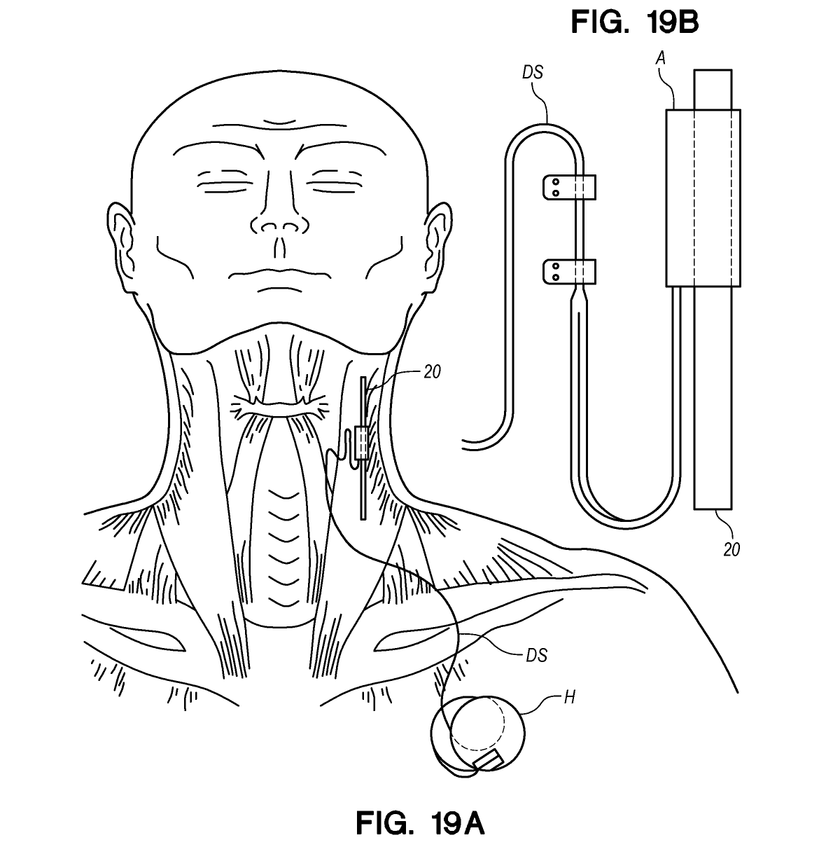

FIG. 19A illustrates an example of a gross anatomical location of an implantation/installation configuration wherein a controller housing (H) is implanted in the chest, and is operatively coupled (via the delivery segment DS) to an applicator (A) positioned to stimulate at least one branch of Vagus Nerve 20. The close-up view of FIG. 19B shows more detail of the exemplary embodiment of Applicator A and its fixation to Vagus Nerve 20. Alternately, Applicator A may be deployed at a more distal nerve branch for the purposes of therapeutic selectivity, and to ameliorate possible side-effects of collateral heating of other vagal nerves, both efferent and afferent.

The electrical connections for devices such as these where the heat source is either embedded within, on, or located nearby to the applicator, may be integrated into the applicators described herein. As described earlier herein, materials like the product sold by NanoSonics, Inc. under the tradename Metal Rubber™ and/or mc10's extensible inorganic flexible circuit platform may be used to fabricate an electrical circuit on or within an applicator, or, alternately, as thermal conduction material. Alternately, the product sold by DuPont, Inc., under the tradename PYRALUX®, or other such flexible and electrically insulating material, like polyimide, may be used to form a flexible circuit; including one with a copper-clad laminate for connections. PYRALUX® in sheet form allows for such a circuit to be rolled. More flexibility may be afforded by cutting the circuit material into a shape that contains only the electrodes and a small surrounding area of polyimide.

Such circuits then may be encapsulated for electrical isolation using a conformal coating. A variety of such conformal insulation coatings are available, including by way of non-limiting example, parlene (Poly-Para-Xylylene) and parlene-C (parylene with the addition of one chlorine group per repeat unit), both of which are chemically and biologically inert. Silicones and polyurethanes may also be used, and may be made to comprise the applicator body, or substrate, itself. The coating material can be applied by various methods, including brushing, spraying and dipping. Parylene-C is a bio-accepted coating for stents, defibrillators, pacemakers and other devices permanently implanted into the body.

In a particular embodiment, biocompatible and bioinert coatings may be used to reduce foreign body responses, such as that may result in cell growth over or around an applicator and change the electrical properties of the system. These coatings may also be made to adhere to the electrodes and to the interface between the array and the hermetic packaging that forms the applicator.

By way of non-limiting example, both parylene-C and poly(ethylene glycol) (PEG, described herein) have been shown to be biocompatible and may be used as encapsulating materials for an applicator. Bioinert materials non-specifically downregulate, or otherwise ameliorate, biological responses. An example of such a bioinert material for use in an embodiment of the present invention is phosphoryl choline, the hydrophilic head group of phospholipids (lecithin and sphingomyelin), which predominate in the outer envelope of mammalian cell membranes. Another such example is Polyethylene oxide polymers (PEO), which provide some of the properties of natural mucous membrane surfaces. PEO polymers are highly hydrophilic, mobile, long chain molecules, which may trap a large hydration shell. They may enhance resistance to protein and cell spoliation, and may be applied onto a variety of material surfaces, such as PDMS, or other such polymers. An alternate embodiment of a biocompatible and bioinert material combination for use in practicing the present invention is phosphoryl choline (PC) copolymer, which may be coated on a PDMS substrate. Alternately, a metallic coating, such as gold or platinum, as were described earlier, may also be used. Such metallic coatings may be further configured to provide for a bioinert outer layer formed of self-assembled monolayers (SAMs) of, for example, D-mannitol-terminated alkanethiols. Such a SAM may be produced by soaking the intended device to be coated in 2 mM alkanethiol solution (in ethanol) overnight at room temperature to allow the SAMs to form upon it. The device may then be taken out and washed with absolute ethanol and dried with nitrogen to clean it.

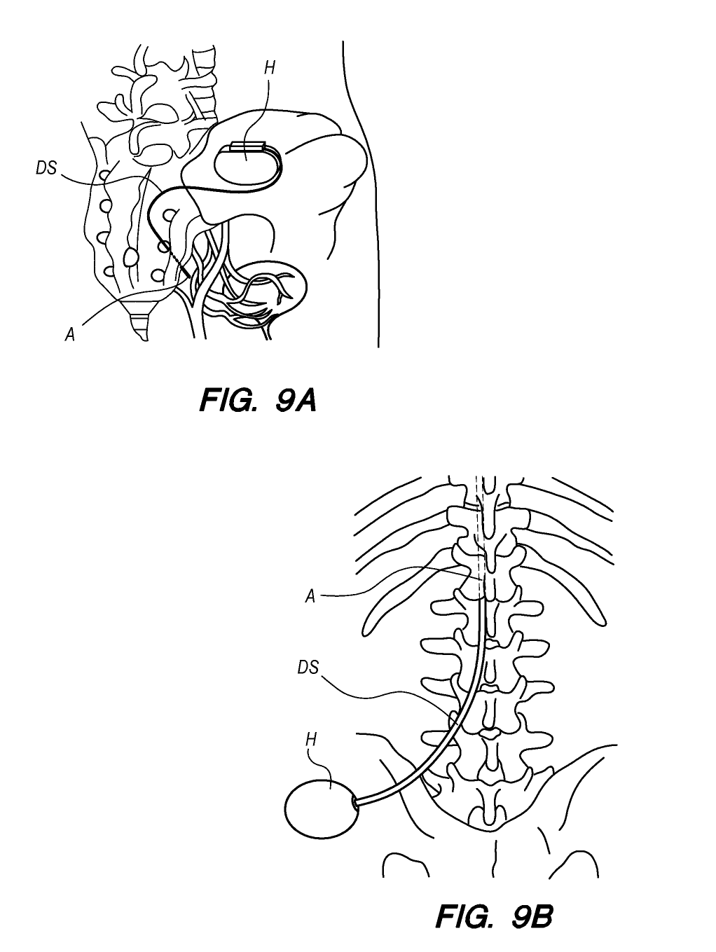

Referring to FIGS. 9A and 9B, two implantation configurations featuring housings (H), placed in different anatomic locations from applicators (A), and operatively coupled thereto by delivery segments (DS) are depicted.

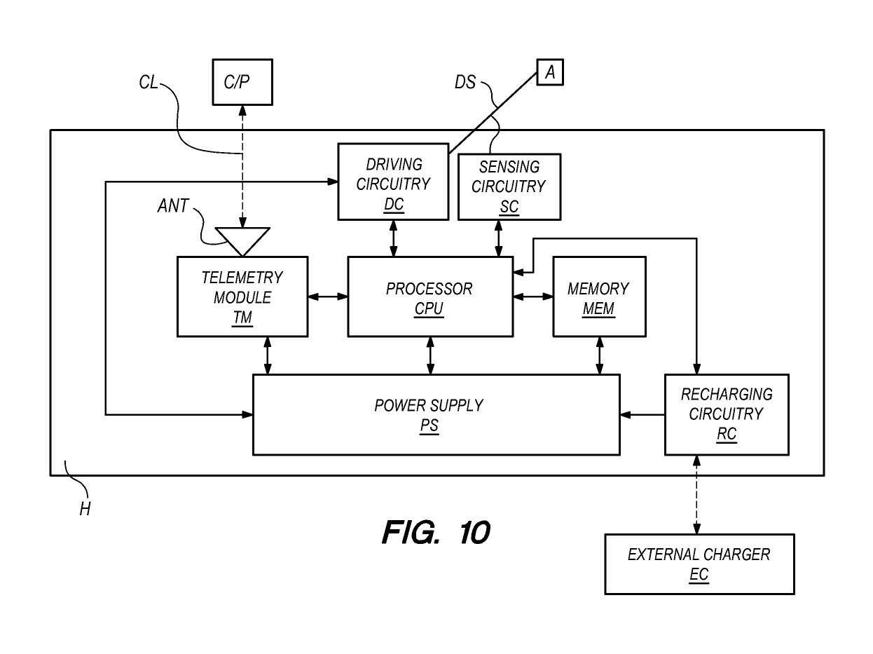

Referring to FIG. 10, a block diagram is depicted illustrating various components of an example implantable housing H. In this example, implantable stimulator includes processor CPU, memory MEM, power supply PS, telemetry module TM, antenna ANT, and the driving circuitry DC for a stimulation generator. As used herein, stimulation refers to heating. The Housing H is coupled to one Delivery Segments DSx, although it need not be. It may be a multi-channel device in the sense that it may be configured to include multiple electrical paths (e.g., multiple heat sources and/or electrical leads) that may deliver different thermal outputs, some of which may have different local target temperatures and/or thermal loads. More or less delivery segments may be used in different implementations, such as, but not limited to, one, two, five or more electrical leads and associated heat sources may be provided. The delivery segments may be detachable from the housing, or be fixed.

Memory (MEM) may store instructions for execution by Processor CPU, temperature sensor data processed by sensing circuitry SC, and obtained from sensors both within the housing, such as battery level, discharge rate, etc., and those deployed outside of the Housing (H), possibly in Applicator A, such as temperature sensors, and/or other information regarding therapy for the patient. Processor (CPU) may control Driving Circuitry DC to deliver power to the heat source (not shown) according to a selected one or more of a plurality of programs or program groups stored in Memory (MEM). Memory (MEM) may include any electronic data storage media, such as random access memory (RAM), read-only memory (ROM), electronically-erasable programmable ROM (EEPROM), flash memory, etc. Memory (MEM) may store program instructions that, when executed by Processor (CPU), cause Processor (CPU) to perform various functions ascribed to Processor (CPU) and its subsystems, such as dictate pulsing parameters for the heat source.

Electrical connections may be through Housing H via an Electrical Feedthrough EFT, such as, by way of non-limiting example, The SYGNUS® Implantable Contact System from Bal-SEAL.

In accordance with the techniques described in this disclosure, information stored in Memory (MEM) may include information regarding therapy that the patient had previously received. Storing such information may be useful for subsequent treatments such that, for example, a clinician may retrieve the stored information to determine the therapy applied to the patient during his/her last visit, in accordance with this disclosure. Processor CPU may include one or more microprocessors, digital signal processors (DSPs), application-specific integrated circuits (ASICs), field-programmable gate arrays (FPGAs), or other digital logic circuitry. Processor CPU controls operation of implantable stimulator, e.g., controls stimulation generator to deliver thermal therapy according to a selected program or group of programs retrieved from memory (MEM). For example, processor (CPU) may control Driving Circuitry DC to deliver electrical signals, e.g., as stimulation pulses, with intensities, pulse durations (if applicable), and rates specified by one or more stimulation programs. Processor (CPU) may also control Driving Circuitry (DC) to selectively deliver the stimulation via subsets of Delivery Segments (DSx), and with stimulation specified by one or more programs. Different delivery segments (DSx) may be directed to different target tissue sites, as was previously described.

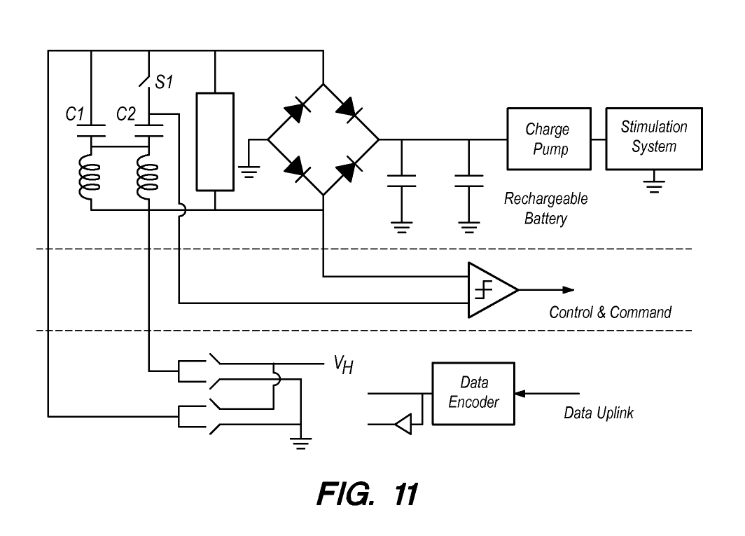

Telemetry module (TM) may include, by way of non-limiting example, a radio frequency (RF) transceiver to permit bi-directional communication between implantable stimulator and each of a clinician programmer module and/or a patient programmer module (generically a clinician or patient programmer, or “C/P”). A more generic form is described above in reference to FIG. 2 as the input/output (I/O) aspect of a controller configuration (P/C). Telemetry module (TM) may include an Antenna (ANT), of any of a variety of forms. For example, Antenna (ANT) may be formed by a conductive coil or wire embedded in a housing associated with medical device. Alternatively, antenna (ANT) may be mounted on a circuit board carrying other components of implantable stimulator or take the form of a circuit trace on the circuit board. In this way, telemetry module (TM) may permit communication with a programmer (C/P). Given the energy demands and modest data-rate requirements, the Telemetry system may be configured to use inductive coupling to provide both telemetry communications and power for recharging, although a separate recharging circuit (RC) is shown in FIG. 10 for explanatory purposes. An alternate configuration is shown in FIG. 11.

Referring to FIG. 11, a telemetry carrier frequency of 175 kHz aligns with a common ISM band and may use on-off keying at 4.4 kbps to stay well within regulatory limits. Alternate telemetry modalities are discussed elsewhere herein. The uplink may be an H-bridge driver across a resonant tuned coil. The telemetry capacitor, C1, may be placed in parallel with a larger recharge capacitor, C2, to provide a tuning range of 50-130 kHz for optimizing the RF-power recharge frequency. Due to the large dynamic range of the tank voltage, the implementation of the switch, S1, employs a nMOS and pMOS transistor connected in series to avoid any parasitic leakage. When the switch is OFF, the gate of pMOS transistor is connected to battery voltage, V_{Battery}, r and the gate of nMOS is at ground. When the switch is ON, the pMOS gate is at negative battery voltage, −V_{Battery}, and the nMOS gate is controlled by charge pump output voltage. The ON resistance of the switch is designed to be less than 5Ω to maintain a proper tank quality factor. A voltage limiter, implemented with a large nMOS transistor, may be incorporated in the circuit to set the full wave rectifier output slightly higher than battery voltage. The output of the rectifier may then charge a rechargeable battery through a regulator.

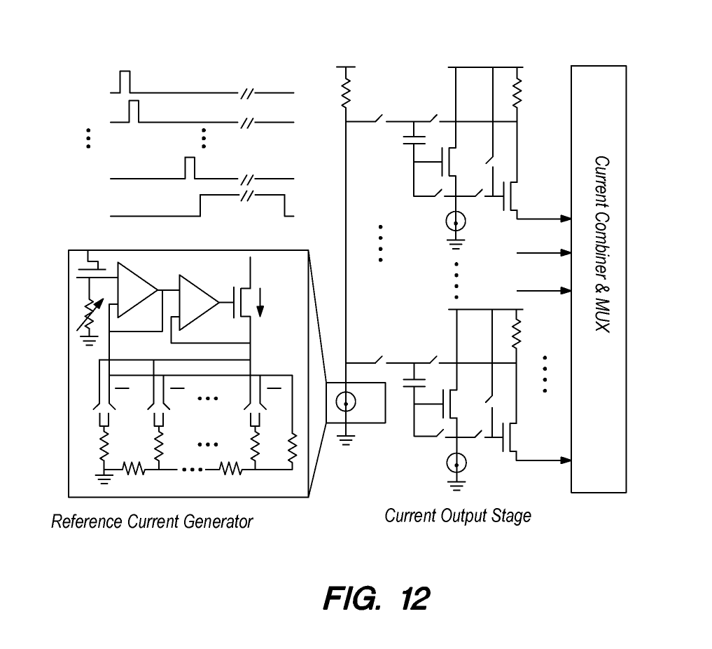

FIG. 12 relates to an embodiment of the Driving Circuitry DC, and may be made to a separate integrated circuit (or “IC”), or application specific integrated circuit (or “ASIC”), or a combination of them.

The control of the output may be managed locally by a state-machine, as shown in this non-limiting example, with parameters passed from the microprocessor. Most of the design constraints are imposed by the output drive DAC. First, a stable current is required to reference for the system. A constant current of 100 nA, generated and trimmed on chip, is used to drive the reference current generator, which consists of an R-2R based DAC to generate an 8-bit reference current with a maximum value of 5 μA. The reference current may then amplified in the current output stage with the ratio of Rₒ and R_{ref}, designed as a maximum value of 40, for example. An on-chip sense-resistor-based architecture may be used for the current output stage to eliminate the need to keep output transistors in saturation, reducing voltage headroom requirements to improve power efficiency. The architecture may use thin-film resistors (TFRs) in the output driver mirroring to enhance matching. To achieve accurate mirroring, the nodes X and Y may be forced to be the same by the negative feedback of the amplifier, which results in the same voltage drop on Rₒ and R_{ref}. Therefore, the ratio of output current, Iₒ, and the reference current, I_{ref}, may be made equal to the ratio of and R_{ref} and Rₒ.

The capacitor, C, retains the voltage acquired in the precharge phase. When the voltage at Node Y is exactly equal to the earlier voltage at Node X, the stored voltage on C biases the gate of P2 properly so that it balances I_{bias}. If, for example, the voltage across Rₒ is lower than the original R_{ref} voltage, the gate of P2 is pulled up, allowing I_{bias} to pull down on the gate on P1, resulting in more current to R_{O}. In the design of this embodiment, charge injection may be minimized by using a large holding capacitor of 10 pF. The performance may be eventually limited by resistor matching, leakage, and finite amplifier gain. With 512 current output stages, the heater drive IC may drive two outputs for separate heaters (as shown in FIG. 12) with separate sources, each delivering a maximum current of 51.2 mA.

Alternatively, if the maximum back-bias on the thermal generating element can withstand the drop of the other element, then the devices can be driven in opposite phases (one as sinks, one as sources) and the maximum current exceeds 100 mA. The stimulation rate can be tuned from about 0.01 Hz to about 1 kHz and the pulse or burst duration(s) can be tuned from about 100s to about 1 ms. However, the actual limitation in the stimulation output pulse-train characteristic is ultimately set by the energy transfer of the charge pump, and this generally should be considered when configuring the therapeutic protocol. Similarly, it may be made to monitor the amount of energy delivered in a pulse by controlling one or more of the variable described above, i.e. the current amplitude, pulse duration, pulse interval, and the treatment duty cycle.

External programming devices for patient and/or physician can be used to alter the settings and performance of the implanted housing. Similarly, the implanted apparatus may communicate with the external device to transfer information regarding system status and feedback information. This may be configured to be a PC-based system, or a stand-alone system. In either case, the system generally should communicate with the housing via the telemetry circuits of Telemetry Module (TM) and Antenna (ANT). Both patient and physician may utilize controller/programmers (C/P) to tailor stimulation parameters such as duration of treatment, voltage or amplitude, pulse duration, pulse frequency, burst length, and burst rate, as is appropriate.

Once the communications link (CL) is established, data transfer between the MMN programmer/controller and the housing may begin. Examples of such data are:

1. From housing to controller/programmer:
a. Patient usage
b. Battery lifetime
c. Feedback data
   i. Device diagnostics (such as target and/or internal temperature)

2. From controller/programmer to housing:
a. Updated temperature and/or output power level settings based upon device diagnostics
b. Alterations to pulsing scheme
c. Reconfiguration of embedded circuitry
   i. such as field programmable gate array (FPGA), application specific integrated circuit (ASIC), or other integrated or embedded circuitry

By way of non-limiting examples, near field communications, either low power and/or low frequency may be employed for telemetry. In 2009 (and then updated in 2011), the US FCC dedicated a portion of the EM Frequency spectrum for the wireless biotelemetry in implantable systems, known as The Medical Device Radiocommunications Service (known as “MedRadio” and also known as Medical Implant Communication Service or “MICS”). Devices employing such telemetry may be known as “medical micropower networks” or “MMN” services. The currently reserved spectra are in the 401-406, 413-419, 426-432, 438-444, and 451-457 MHz ranges, and provide for certain authorized spectral bands.

Interestingly, these frequency bands are used for other purposes on a primary basis such as Federal government and private land mobile radios, Federal government radars, and remote broadcast of radio stations. It has recently been shown that higher frequency ranges are also applicable and efficient for telemetry and wireless power transfer in implantable medical devices. MICS chipsets are available from MicroSemi, Inc., such as the Zarlink ZL70321 mixed-band, low-power radio.

An MMN may be made not to interfere or be interfered with by external fields by means of a magnetic switch in the implant itself. Such a switch may be only activated when the MMN programmer/controller is in close proximity to the implant. This also provides for improved electrical efficiency due to the restriction of emission only when triggered by the magnetic switch. Giant Magnetorestrictive (GMR) devices are available with activation field strengths of between 5 and 150 Gauss. This is typically referred to as the magnetic operate point. There is intrinsic hysteresis in GMR devices, and they also exhibit a magnetic release point range that is typically about one-half of the operate point field strength. Thus, a design utilizing a magnetic field that is close to the operate point will suffer from sensitivities to the distance between the housing and the MMN programmer/controller, unless the field is shaped to accommodate this. Alternately, one may increase the field strength of the MMN programmer/controller to provide for reduced sensitivity to position/distance between it and the implant. In a further embodiment, the MMN may be made to require a frequency of the magnetic field to improve the safety profile and electrical efficiency of the device, making it less susceptible to errant magnetic exposure. This can be accomplished by providing a tuned electrical circuit (such as an L-C or R-C circuit) at the output of the switch.

Alternately, another type of magnetic device may be employed as a switch. By way of non-limiting example, a MEMS device may be used. A cantilevered MEMS switch may be constructed such that one member of the MEMS may be made to physically contact another aspect of the MEMS by virtue of its magnetic susceptibility, similar to a miniaturized magnetic reed switch. The suspended cantilever may be made to be magnetically susceptible by depositing a ferromagnetic material (such as, but not limited to Ni, Fe, Co, NiFe, and NdFeB) atop the end of the supported cantilever member. Such a device may also be tuned by virtue of the cantilever length such that it only makes contact when the oscillations of the cantilever are driven by an oscillating magnetic field at frequencies beyond the natural resonance of the cantilever.

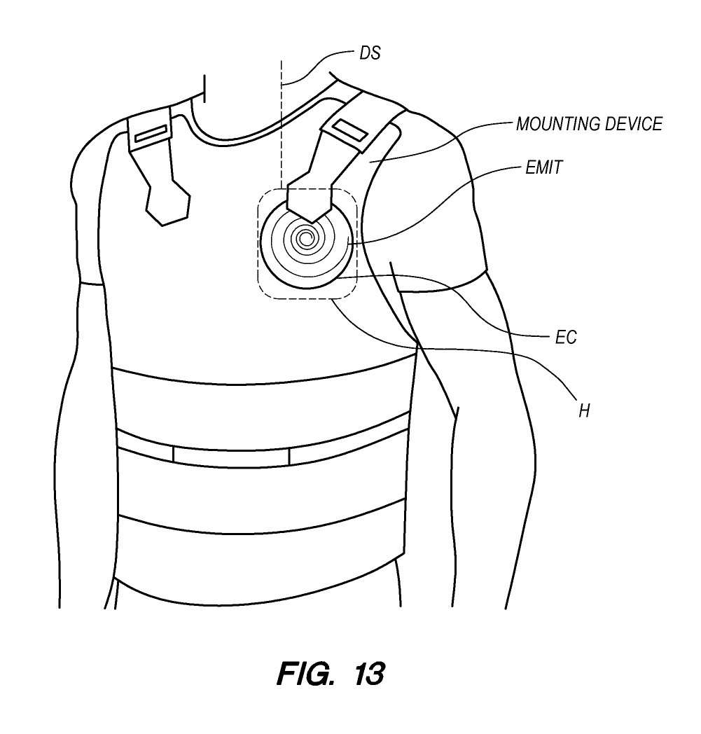

FIG. 13 illustrates an embodiment, where an external charging device is mounted onto clothing for simplified use by a patient, comprising a Mounting Device MOUNTING DEVICE, which may be selected from the group consisting of, but not limited to: a vest, a sling, a strap, a shirt, and a pant. Mounting Device MOUNTING DEVICE further comprising a Wireless Power Transmission Emission Element EMIT, such as, but not limited to, a magnetic coil, or electrical current carrying plate, that is located substantially nearby an implanted power receiving module, such as is represented by the illustrative example of Housing H, which is configured to be operatively coupled to Delivery Segment(s) DS. Within Housing H, may be a power supply, and controller, such that the controller activates the heat source by controlling current thereto. Alternately, the power receiving module may be located at the applicator (not shown).

Alternately, a system may be configured to utilize one or more wireless power transfer inductors/receivers that are implanted within the body of a patient that are configured to supply power to the implantable power supply.

There are a variety of different modalities of inductive coupling and wireless power transfer. For example, there is non-radiative resonant coupling, such as is available from Witricity, or the more conventional inductive (near-field) coupling seen in many consumer devices. All are considered within the scope of the present invention. The proposed inductive receiver may be implanted into a patient for a long period of time. Thus, the mechanical flexibility of the inductors may need to be similar to that of human skin or tissue. Polyimide that is known to be biocompatible was used for a flexible substrate.

By way of non-limiting example, a planar spiral inductor may be fabricated using flexible printed circuit board (FPCB) technologies into a flexible implantable device. There are many kinds of a planar inductor coils including, but not limited to; hoop, spiral, meander, and closed configurations. In order to concentrate a magnetic flux and field between two inductors, the permeability of the core material is the most important parameter. As permeability increases, more magnetic flux and field are concentrated between two inductors. Ferrite has high permeability, but is not compatible with microfabrication technologies, such as evaporation and electroplating. However, electrodeposition techniques may be employed for many alloys that have a high permeability. In particular, Ni (81%) and Fe (19%) composition films combine maximum permeability, minimum coercive force, minimum anisotropy field, and maximum mechanical hardness. An exemplary inductor fabricated using such NiFe material may be configured to include 200 μm width trace line width, 100 μm width trace line space, and have 40 turns, for a resultant self-inductance of about 25 μH in a device comprising a flexible 24 mm square that may be implanted within the tissue of a patient. The power rate is directly proportional to the self-inductance.

The radio-frequency protection guidelines (RFPG) in many countries such as Japan and the USA recommend the limits of current for contact hazard due to an ungrounded metallic object under the electromagnetic field in the frequency range from 10 kHz to 15 MHz. Power transmission generally requires a carrier frequency no higher than tens of MHz for effective penetration into the subcutaneous tissue.

In certain embodiments of the present invention, an implanted power supply may take the form of, or otherwise incorporate, a rechargeable micro-battery, and/or capacitor, and/or super-capacitor to store sufficient electrical energy to operate the heat source and/or other circuitry within or associated with the implant when used along with an external wireless power transfer device. Exemplary microbatteries, such as the Rechargeable NiMH button cells available from VARTA, are within the scope of the present invention. Supercapacitors are also known as electrochemical capacitors.

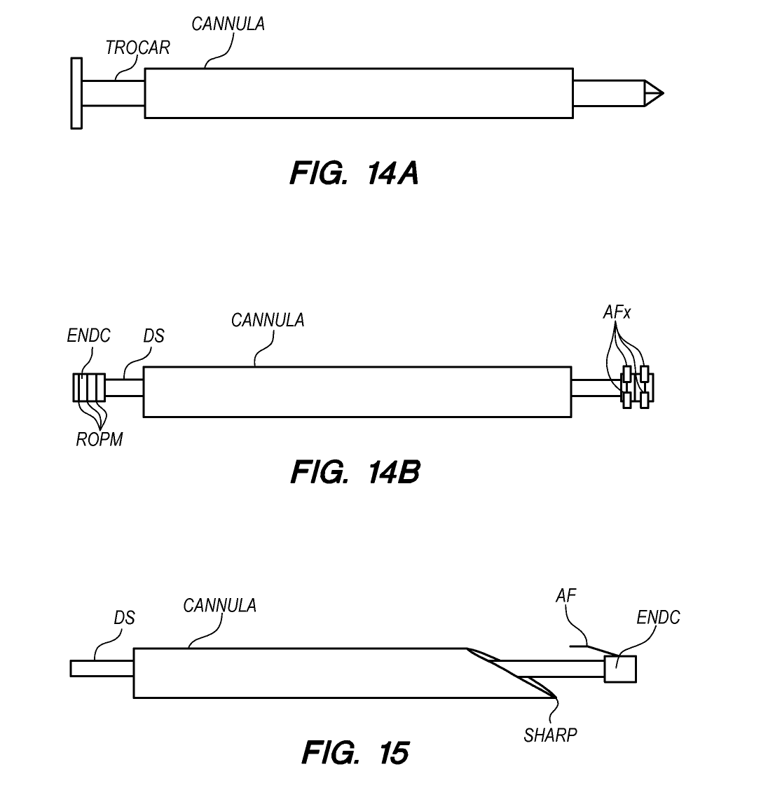

FIGS. 14A and 14B show an alternate embodiment of the present invention, where a Trocar and Cannula may be used to deploy an at least partially implantable system for thermal mediation of the vagus nerve for the control of cough. Trocar TROCAR may be used to create a tunnel through tissue between surgical access points that may correspond to the approximate intended deployment locations of elements of the present invention, such as applicators and housings. Cannula CANNULA may be inserted into the tissue of the patient along with, or after the insertion of the trocar. The trocar may be removed following insertion and placement of the cannula to provide an open lumen for the introduction of system elements. The open lumen of cannula CANNULA may then provide a means to locate delivery segment DS along the route between a housing and an applicator. The ends of delivery segment DS may be covered by end caps ENDC. End caps ENDC may be further configured to comprise radio-opaque markings ROPM to enhance the visibility of the device under fluoroscopic imaging and/or guidance. End Caps ENDC may provide a watertight seal to ensure that the electrical contacts of the Delivery Segment DS, or other system component being implanted, are not degraded. The cannula may be removed subsequent to the implantation of delivery segment DS. Subsequently, delivery segment DS may be connected to an applicator that is disposed to the target tissue and/or a housing, as have been described elsewhere herein. In a further embodiment, the End Caps ENDC, or the Delivery Segment DS itself may be configured to also include a temporary Tissue Fixation elements AFx, such as, but not limited to; hook, tines, and barbs, that allow the implanted device to reside securely in its location while awaiting further manipulation and connection to the remainder of the system.

FIG. 15 illustrates an alternate embodiment, similar to that of FIGS. 14A&B, further configured to utilize a barbed Tissue Fixation Element AF that is affixed to End Cap ENDC. Tissue Fixation Element AF may be a barbed, such that it will remain substantially in place after insertion along with Cannula CANNULA, shown in this example as a hypodermic needle with sharp End SHARP being the leading end of the device as it is inserted into a tissue of a patient. The barbed feature(s) of Tissue Fixation Element AF insert into tissue, substantially disallowing Delivery Segment DS to be removed. In a still further embodiment, Tissue Fixation Element AF may be made responsive to an actuator, such as a trigger mechanism (not shown) such that it is only in the configuration to affirmatively remain substantially in place after insertion when activated, thus providing for the ability to be relocated more easily during the initial implantation, and utilized in conjunction with a forward motion of Delivery Segment DS to free the end from the tissue it has captured. Delivery Segment DS may be substantially inside the hollow central lumen of Cannula CANNULA, or substantially slightly forward of it, as is shown in the illustrative embodiment. As used herein, cannula also refers to an elongate member, or delivery conduit. The elongate delivery conduit may be a cannula. The elongate delivery conduit may be a catheter. The catheter may be a steerable catheter. The steerable catheter may be a robotically steerable catheter, configured to have electromechanical elements induce steering into the elongate delivery conduit in response to commands made by an operator with an electronic master input device that is operatively coupled to the electromechanical elements. The surgical method of implantation further may comprise removing the elongate delivery conduit, leaving the delivery segment in place between the first anatomical location and the second anatomical location.

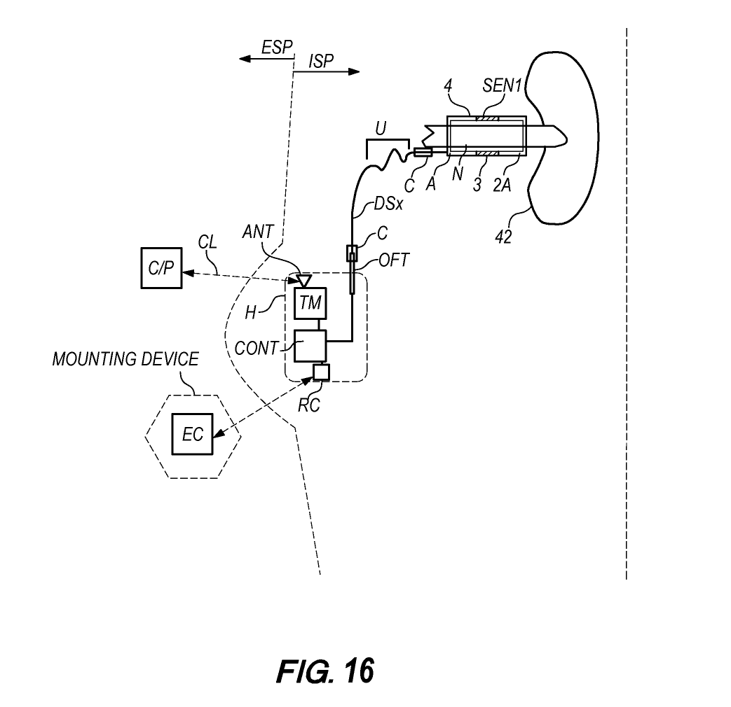

FIG. 16 shows an alternate exemplary embodiment of a system for the treatment of cough via thermal inhibition of the vagus nerve, comprising elements, such as has been described herein. Applicator A, a rolled slab-type applicator that is 12 mm wide and 15 mm long when unrolled, such as has been described herein is deployed about the Target Tissue N, which contains Afferent Nerve(s) 52, of Lung 42. Applicator A further comprises Sensor SEN1, such as has been described herein. Electrical energy is delivered to Applicator A via Delivery Segments DS to produce heat within Applicator A. Connector C is configured to operatively couple electrical energy from Delivery Segments DS to Applicator A, such as has been described herein. Electrical Lead(s) 88 resident within Delivery Segments DS may be connected to the Controller CONT of Housing H via an Electrical Feedthrough EFT, such as, by way of non-limiting example, The SYGNUS® Implantable Contact System from Bal-SEAL. Delivery Segments DS further comprise Undulations U, such as has been described herein. Delivery Segments DS are further configured to comprise Signal Wires SW between Sensor SEN1 and the Controller CONT of Housing H. Delivery Segments DS are operatively coupled to Housing H via Connector C, such as has been described herein The Controller CONT shown within Housing H is a simplification, for clarity, of that described herein. Sensor(s) SEN1 may be a thermocouple, RTD, or other such thermal sensor as has been described herein. External clinician programmer module and/or a patient programmer module C/P may communicate with Controller CONT via Telemetry module TM via Antenna ANT via Communications Link CL, such as has been described herein. Power Supply PS, not shown for clarity, may be wirelessly recharged using External Charger EC, such as has been described herein. Furthermore, External Charger EC may be configured to reside within a Mounting Device MOUNTING DEVICE, such as has been described herein. Mounting Device MOUNTING DEVICE may be a vest, as is especially well configured for this exemplary embodiment. External Charger EC, as well as External clinician programmer module and/or a patient programmer module C/P and Mounting Device MOUNTING DEVICE may be located within the extracorporeal space ESP, while the rest of the system is implanted and may be located within the intracorporeal space ISP, such as has been described herein. External clinician programmer module and/or a patient programmer module C/P may be configured, in conjunction with Controller CONT to provide treatment in response to a user input, such as a button press. As such, the system may be made to begin treatment on demand, or as deemed needed by a user.

Although not explicitly identified as such, there have been published studies aimed at assessing the potential damage of electrocautery in neurosurgery that effectively describe the Arrhenius molecular damage model, a known standard chemical kinetics relation, wherein the damage may be quantified using a single parameter, Ω, a function of both temperature, T, and time, t, which ranges on the entire positive real axis and is calculated from an Arrhenius integral:

$$Ω ( T , t ) = ln [ \frac{C ( 0 )}{C ( τ )} ] = {\int }_{0}^{τ} A {e}^{- [ \frac{{E}_{a}}{RT ( t )} ]} d t$$

where A is a frequency factor [s⁻¹], τ the total heating time (s), Eₐ an activation energy barrier [J mole⁻¹], R the universal gas constant, 8.3143 [J mole⁻¹ K⁻¹], and T the absolute temperature [K]. The frequency factor, A, and energy barrier, Eₐ, are related to the activation enthalpy and entropy, delta-H* and delta-S*, of the particular reaction of interest. The characteristic behavior of this kinetic model is that below a threshold temperature the rate of damage accumulation is negligible, and it increases precipitously when this value is exceeded. This behavior is to be expected from the exponential temperature dependence of the function. However, it is only linearly time-dependent. Thus, the temperatures employed in hyperthermia of neural tissue may need to be well controlled in order to avoid iatrogenesis due to relatively high rates of damage. For retinal tissue (akin to peripheral nerves) values for A and Eₐ are 10⁹⁹ s⁻¹ and 6×10⁵ J mole⁻¹, respectively.

Temperatures of about 43° C. may be used to inhibit nerve function in living animals, as we have demonstrated. This has been shown to be a safe temperature for long exposure durations, and was not observed by the abovementioned studies to cause significant functional and/or morphological changes. Neural target temperatures ranging from 39° C. to 48° C. provide varying efficacy and safety, and are within the scope of the present invention. Likewise, controlling those temperatures to within ±2° C. via means and methods described elsewhere herein is also within the scope of the present invention. The complete time-temperature history, as described above with regard to the Arrhenius model, is predictive of the amount of damage engendered to molecular constituent of the tissue, both target tissue and the surrounding environment. As such, it may be used in an algorithm for therapeutic dosage.

Published studies by Z. Vujaskovic, et al in Effects of intraoperative hyperthermia on canine sciatic nerve: histopathologic and morphometric studies (Int J Hyperthermia. 1994; 10(6):845-55), J. Wondergem, et al in Effects of Local Hyperthermia on the Motor Function of the Rat Sciatic Nerve (doi:10.1080/09553008814552561), and J Carlander, et al in Heat Production, Nerve Function, and Morphology following Nerve Close Dissection with Surgical Instruments (doi:10.1007/s00268-012-1471-x) discuss the Arrhenius-like behavior and the temperature limitations of electrocautery, each is incorporated in their entirety by reference.

The

Experimental Confirmation:

A guinea pig was anesthetized with katamine and xylazine (IM) and laid supine. The ventral neck was shaved and cleaned. An incision was made in the neck and the trachea carefully isolated. An incision was made in the trachea near the carina, and a cannula (breathing tube) inserted into the trachea. The breathing tube was connected via a T-connector to a pressure transduced to measure pressure changes within the breathing tube that corresponded to breathing and coughing. The end of the breathing tube was placed in a chamber filled with humidified 37 degree C. air. The section of the trachea rostral of the cannula was opened and superfused with 37 degree C. Krebs Henseleit buffer. To elicit cough, 100 μL aliquots of citric acid was placed on the superfused trachea. The cough responses were recorded as both changes in respiratory pressure and visually as exaggerated abdominal contractions.

Both vagi were carefully dissected clear of adjacent tissues, including the carotid arteries. Cuffs were placed around the vagi and the heating elements within the cuffs were connected to a power supply. Embedded in the cuffs were thermocouples to record the temperatures within the cuffs. By varying the current supplied by the power supply to the cuffs fine control of the temperature within the cuffs could be accomplished.

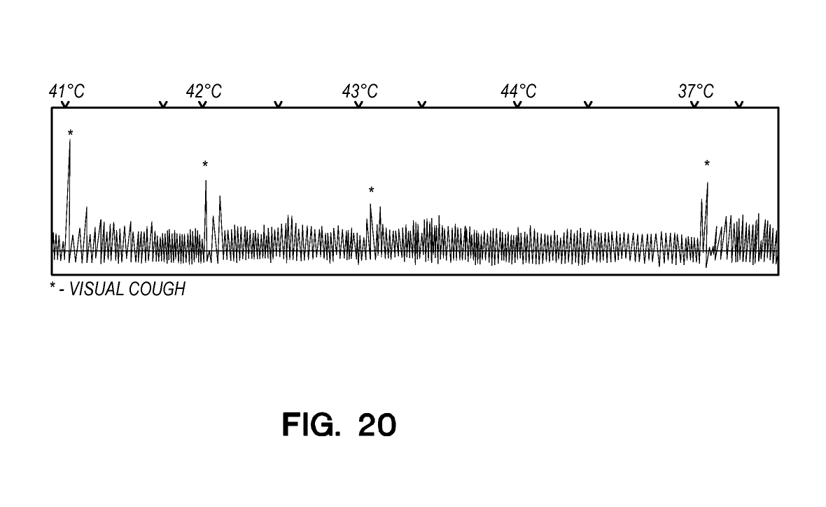

As seen in FIG. 20, increasing the temperature from 41 degrees C. to 44 degrees C. showed a diminished cough response to citric acid applied to the surface of the trachea. At 44 degrees C. the response is completely inhibited. When the temperature was allowed to return to 37 degrees C. the response to citric acid was fully restored. Thus showing that increasing the temperature of the vagus nerves to 44 degrees C. can inhibit the cough reflex and that this effect is completely reversible when the temperature of the nerve returns to normal body temperature.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

1. A system for managing unproductive cough in a patient, comprising:
a. an applicator comprising a resistive heating element and being configured to be positioned adjacent a portion of a targeted nerve tissue for treatment;
b. a power source configured to provide electrical current to the resistive heating element; and
c. a current controller operatively coupled to the power source and configured to raise the temperature of the portion of the targeted nerve tissue to inhibit nerve conduction.

2. The system of claim 1, wherein the targeted nerve tissue comprises at least one vagal afferent nerve.

3. The system of claim 2, wherein the resistive heating element is configured to be positioned immediately adjacent to the at least one vagal afferent nerve.

4. The system of claim 2, wherein the resistive heating element is configured to at least partially surround the at least one vagal afferent nerve.

5. The system of claim 1, wherein the applicator further comprises a temperature sensor operatively coupled to the current controller.

6. The system of claim 5, wherein the temperature sensor is configured to produce an electrical signal representative of a nearby temperature and deliver the electrical signal to the current controller, the current controller being configured to vary the electrical current provided to the resistive heating element based at least in part upon the electrical signal from the temperature sensor.

7. The system of claim 6, wherein the current controller is configured to maintain the temperature of the targeted nerve tissue portion within a desired range for a period of time.

8. The system of claim 5, wherein the temperature sensor comprises a sensor selected from the group consisting of: a bimetallic sensor or switch, a fluid expansion sensor or switch, a thermocouple, a thermistor, a Resistance Temperature Detector, and an infrared pyrometer.

9. The system of claim 7, wherein the desired range is between about 38 degrees Celsius and about 46 degrees Celsius.

10. The system of claim 7, wherein the desired range is within ±2 degrees Celsius of a nominal temperature within a range of about 38 degrees Celsius and about 46 degrees Celsius.

11. The system of claim 1, wherein the applicator further comprises an electrical activity sensor operatively coupled to the current controller and configured to produce an electrical signal representative of electrical activity of at least one nerve.

12. The system of claim 11, wherein the controller is configured to interpret the signal from the electrical activity sensor and vary the current to the resistive heating element at least in part relative to the electrical signal representative of electrical activity of the at least one nerve.

13. The system of claim 12, wherein the controller is configured to maintain a level of activity of the targeted nerve tissue portion within a desired range for a period of time.

14. The system of claim 13, wherein the controller is operatively coupled to a temperature sensor and is configured to also maintain a temperature of the targeted nerve tissue portion within a desired range for a period of time.

15. The system of claim 1, wherein the current controller is further configured to deliver the electrical current in a pulsatile fashion.

16. The system of claim 15, wherein the pulsatile fashion comprises current pulses delivered have a time duration between about 1 millisecond and about 100 seconds.

17. The system of claim 15, wherein the pulsatile fashion comprises a current pulse duty cycle of between about 99% and 0.1%.

18. The system of claim 15, wherein the current controller further is configured to be controlled for an output characteristic selected from the group consisting of: current amplitude, pulse duration, duty cycle, and overall energy delivered.

19. The system of claim 1, wherein the current controller is configured to be responsive to at least one patient input.

20. The system of claim 19, wherein the current controller is configured such that the at least one patient input triggers a delivery of current to the resistive heating element.

21. The system of claim 4, wherein the applicator is placed to at least 60% circumferentially surround a vagal afferent nerve or vagal afferent nerve bundle.
